(19) United States
(12) Patent Application Publication
(10) Pub. No.: US 2006/0204999 A1
(43) Pub. Date: Sep. 14, 2006

(54) Detecting molecular complexes

(76) Inventor: Stephen MacEvicz, Cupertino, CA (US)

Correspondence Address: STEPHEN C. MACEVICZ, 21890 RUCKER DRIVE, CUPERTINO, CA, 95014, US

(21) Appl. No.: 11/374,506

(22) Filed: Mar. 13, 2006

Related U.S. Application Data
(60) Provisional application No. 60/708,989, filed on Aug. 17, 2005.
(60) Provisional application No. 60/662,036, filed on Mar. 14, 2005.

(51) Int. Cl. C12Q 1/68; C12P 19/34

(52) U.S. Cl. 435/6; 435/91.2

(57) ABSTRACT

The invention provides methods and compositions for detecting the presence of, or for measuring amounts of, molecular complexes, particularly complexes comprising two or more proteins, such as receptor complexes of cell surface membranes. In one aspect of the invention, reagent pairs are provided that comprise a cleaving probe that specifically binds to at least one component of a molecular complex and one or more signaling reagents that specifically bind to one or more components of the molecular complex, at least one of which is different from the component to which the cleaving probe is attached. Each signaling reagent comprises a binding compound specific for a component of the molecular complex and a signaling polynucleotide attached thereto by a cleavable linkage. When the reagent pairs are bound to the same molecular complex, the cleaving probe may be induced to generate a reactive species that is capable of cleaving cleavable linkages within an effective proximity, thereby releasing a signaling polynucleotide by which the molecular complex is detected.

This application claims priority to U.S. provisional patent applications Ser. No. 60/708,989 filed 17 Aug. 2005 and Ser. No. 60/662,036 filed 14 Mar. 2005, both of which are incorporated by reference.

### FIELD OF THE INVENTION

The present invention relates generally to assays for biomolecules, and more particularly, to the detection and measurement of complexes of biomolecules.

### BACKGROUND OF THE INVENTION

The formation and disassociation of molecular complexes is a pervasive biological phenomenon that is crucial to regulatory processes in living organisms. For example, the interactions of several cell surface membrane components play crucial roles in transmitting extracellular signals to a cell in normal physiology, and in disease conditions. For example, cell surface receptors frequently undergo dimerization, oligomerization, or clustering in connection with the transduction of an extracellular event, such as ligand-receptor binding, into a cellular response, such as proliferation, increased or decreased gene expression, or the like, e.g. George et al, Nature Reviews Drug Discovery, 1: 808-820 (2002); Mellado et al, Ann. Rev. Immunol., 19: 397-421 (2001); Schlessinger, Cell, 103: 211-225 (2000); Yarden, Eur. J. Cancer, 37: S3-S8 (2001). The role of such signal transduction events in diseases, such as cancer, has been the object of intense research and has led to the development of several new drugs and drug candidates, e.g. Herbst and Shin, Cancer, 94: 1593-1611 (2002); Yarden and Sliwkowski, Nature Reviews Molecular Cell Biology, 2: 127-137 (2001); McCormick, Trends in Cell Biology, 9: 53-56 (1999); Blume-Jensen and Hunter, Nature, 411: 355-365 (2001); Baselga, Cancer Cell, 2: 93-95 (2002); Agus et al, Cancer Cell, 2: 127-137 (2002); Koll et al, International patent publication WO 2004/008099.

In addition to their critical role in a host of biological processes, protein-protein complexes may be associated with particular disease conditions, such as antibody-mediated pure red cell aplasia, or PRCA. This condition may arise in patients being treated with recombinant erythropoietin (EPO), which in some cases elicits a patient antibody response that produces neutralizing antibodies against EPO, e.g. Bennett et al, New England J. Medicine, 351: 1403-1408 (2005); Casadevall, Nephrol. Dial. Transplant., 20 (Suppl. 4) iv3-iv8 (2005); Macdougall, Nephrol. Dial. Transplant., 20 (Suppl. 4) iv9-iv15 (2005).

A wide variety of techniques have been used to study cellular protein-protein interactions and complexes, including immunoprecipitation, chemical cross-linking, bioluminescence resonance energy transfer (BRET), fluorescence resonance energy transfer (FRET), and the like, e.g. Price et al, Methods in Molecular Biology, 218: 255-267 (2003); McDonald et al, Anal. Biochem., 268: 318-329 (1999); Sorkin et al, Curr. Biol., 10: 1395-1398 (2000); McVey et al, J. Biol. Chem., 17: 14092-14099 (2001); Salim et al, J. Biol. Chem., 277: 15482-15485 (2002); Angers et al, Proc. Natl. Acad. Sci., 97: 3684-3689 (2000); Stagljar, STKE Science 2003, pe56 (2003). Unfortunately, such techniques are frequently difficult to apply, especially in a clinical setting, require relatively large sample sizes, and generally lack sufficient sensitivity to provide an accurate picture of complex molecular interactions, such as those related to signaling pathways. Techniques based on releasable moleucular tags have been proposed for detecting a variety of biological analytes, including molecular complexes; however, such approaches require specialized separation equipment for implementation, and the paucity of performance data related to their applications makes it difficult to evaluation the utility of such approaches for detecting molecular complexes, e.g. Giese, Trends in Anal. Chem., 2: 165-167 (1983); Giese, U.S. Pat. Nos. 4,650,750 and 4,709,016; Chan-Hui et al, International patent publication WO 2004/011900; and Liu et al, U.S. patent publication 2004/0121382.

In view of the above, the availability of a convenient, sensitive, and cost effective technique for simultaneously detecting or measuring one or more molecular complexes, particularly those in signaling pathways, would advance many fields where such measurements are becoming increasingly important, such as biomedical research, diagnostics, drug discovery, and the like.

### SUMMARY OF THE INVENTION

In one aspect, the invention provides methods and compositions for detecting the presence of, or for measuring amounts of, molecular complexes, particularly complexes comprising two or more proteins, such as receptor complexes of cell surface membranes. In one aspect of the invention, reagent pairs are provided that comprise a cleaving probe that specifically binds to at least one component of a molecular complex and one or more signaling reagents that specifically bind to one or more components of the molecular complex, at least one of which is different from the component to which the cleaving probe is attached. Each signaling reagent comprises a binding compound specific for a component of the molecular complex and a signaling polynucleotide attached thereto by a cleavable linkage. When the reagent pairs are bound to the same molecular complex, the cleaving probe may be induced to generate a reactive species that is capable of cleaving cleavable linkages within an effective proximity, thereby releasing a signaling polynucleotide. The signaling polynucleotide may be isolated from non-released signaling polynucleotides in order to generate a signal, e.g. by amplification, or non-released signaling polynucleotides may be rendered incapable of generating a signal, in which case no isolation is necessary.

In another aspect, the invention provides reagents and methods for homogeneous assays for molecular complexes, such assays further including signaling polynucleotide that may be circularized in a template-driven reaction after release for the purpose of protecting them from exonuclease digestion of non-released signaling polynucleotides. After such digestion, circularized signaling polynucleotides may be detected by a variety of amplication-detection techniques.

In still another aspect, the invention provide methods and compounds for measuring relative phosphorylation states of one or more proteins. In a preferred embodiment of this aspect, triplets of binding compounds are provided for each phosphoprotein to be detected: (i) a first binding compound specific for a first antigenic determinant, the first binding compound having a first polynucleotide attached thereto by a cleavable linkage, the first polynucleotide have a first oligonucleotide tag; (ii) a second binding compound specific for a second antigenic determinant, the second binding compound having a second polynucleotide attached thereto by a cleavable linkage, the second polynucleotide have a second oligonucleotide tag; and (iii) a cleaving probe specific for a third antigenic determinant, the cleaving probe having a cleaving agent capable of generating a reactive species within an effective proximity, and the reactive species being capable of cleaving the cleavable linkage. Each of the first, second, and third antigenic determinants are different, and preferably non-overlapping, and at least one of the first or second binding compounds binds to an antigenic determinant that includes a phosphate group, such that substantially no binding occurs whenever such phosphate group is absent. Such aspect further includes a method of detecting multiple phosphoproteins by the following steps: (i) providing the above binding compounds and cleaving probe; (ii) combining with the sample in an assay mixture the binding compounds and the cleaving probe so that the binding compounds and cleaving probe specifically bind to their respective antigenic determinants whenever present, and so that the cleavable linkages of the binding compounds are within the effective proximity of the cleaving agent of the cleaving probe and are cleaved by the reactive species of such agent, and the polynucleotides of the binding compounds are released; and (iv) determining the presence and phosphorylation status of the phosphoproteins by the released polynucleotides. Preferably, such polynucleotides are determined by way of their oligonucleotide tags. In one aspect, such oligonucleotide tags are determined by amplification, such as by real-time PCR. In another aspect, such oligonucleotide tags are determined by hybridization to their respective complements on one or more solid phase supports.

In one aspect, the method of the invention is carried out with the following steps: (i) providing a binding compound specific for the first component, the binding compound having a polynucleotide attached thereto by a cleavable linkage; (ii) providing a cleaving probe specific for the second component, the cleaving probe having a cleaving agent capable of generating a reactive species within an effective proximity, and the reactive species being capable of cleaving the cleavable linkage; (iii) combining with the sample in an assay mixture the binding compound and the cleaving probe so that the binding compound and cleaving probe specifically bind to the first and second components, respectively, and so that whenever the first component and the second component are in a molecular complex, the cleavable linkage is within the effective proximity of the cleaving agent and are cleaved by the reactive species, and the polynucleotide is released; and (iv) determining the presence or amount of the molecular complex by the released polynucleotide.

In another aspect, the method of the invention is carried out with the following steps: (i) providing a binding compound specific for the first component, the binding compound having a polynucleotide attached thereto by a cleavable linkage, the polynucleotide comprising a first end, an oligonucleotide tag, a second end, and a complementary functionality at the first end or the second end, and the cleavable linkage being attached to the polynucleotide by an end opposite to that of the complementary functionality, and upon cleavage generating on such end a reactive functionality; (ii) providing a cleaving probe specific for the second component, the cleaving probe having a cleaving agent capable of generating a reactive species within an effective proximity, and the reactive species being capable of cleaving the cleavable linkage; (iii) combining with the sample in an assay mixture the binding compound and the cleaving probe so that the binding compound and cleaving probe specifically bind to the first and second components, respectively, and so that whenever the first component and the second component are in a molecular complex, the cleavable linkage is within the effective proximity of the cleaving agent and are cleaved by the reactive species, and the polynucleotide is released; (iv) circularizing the released polynucleotide by reacting the functionality with the complementary functionality in a template-driven reaction wherein the first end of the polynucleotide and the second end of the polynucleotide hybridize to a template oligonucleotide complementary thereto; (v) digesting uncircularized polynucleotide; and (vi) determining the presence or amount of the molecular complex by the oligonucleotide tags of the circularized polynucleotides.

In one aspect, compositions of the invention comprise the following components: a plurality of binding compounds each having a polynucleotide attached by a cleavable linkage and each having a specificity for an antigen, such that each different binding compound has a different polynucleotide attached, and such that a binding compound having specificity for a different antigen has a different polynucleotide attached.

In another aspect, compositions of the invention comprise: (a) one or more binding compounds each having a polynucleotide attached by a cleavable linkage and each having a specificity for a molecular complex, such that each different binding compound has a different polynucleotide attached, and such that a binding compound having specificity for a different antigenic determinant of the molecular complex has a different polynucleotide attached; and (b) a cleaving probe specific for the molecular complex, the cleaving probe having a cleaving agent for generating an reactive species for cleaving the cleavable linkage.

In still another aspect, the invention provide a nonhomogeneous assay where signaling probes are captured on a solid support by an interaction of non-nucleic acid components. In one embodiment, such interaction is the affinity capture of antibody-containing reagents by protein A or protein G, or like capture agent. Thus, released oligonucleotide tags are not captured and can be eluted from the reaction mixture for amplification and detection.

In another aspect, methods and compositions of the invention are used to detect immunogenic responses in patients being treated with protein therapeutics, such as therapeutic antibodies or recombinant forms of natural proteins, such as erythropoietin.

### BRIEF DESCRIPTION OF THE DRAWINGS

FIG. 1 illustrates diagrammatically a signaling reagent and cleaving probe of the invention and release of a signaling polynucleotide.

FIGS. 2A-2B illustrate diagrammatically circularization of a released signaling polynucleotide.

FIGS. 3A-3B illustrate diagrammatically digestion of non-circularized signaling polynucleotides and amplification of circularized signaling polynucleotides for detection.

FIGS. 4A-4E illustrate diagrammatically a non-homogeneous assay format using biotinylated beads as capture agents.

FIGS. 5A-5C illustrate diagrammatically a non-homogeneous assay format using a support derivatized with protein A as a capture agent.

FIGS. 6A-6C illustrate diagrammatically a non-homogeneous assay format for measuring human anti-therapeutic protein antibodies.

### DEFINITIONS

“14-3-3 protein” means a human protein capable of forming a stable complex with a human BAD phosphorylated at Ser-112 and/or Ser-155, such protein having an amino acid sequence substantially identical to that described under NCBI accession number AAH56867 or in Strausberg et al, Proc. Natl. Acad. Sci., 99: 16899-16903 (2002). In one aspect, a 14-3-3 protein hereunder is at least eighty percent identical, and more preferably ninety percent identical, to the amino acid described under NCBI accession number AAH56867 or in Strausberg et al, Proc. Natl. Acad. Sci., 99: 16899-16903 (2002).

“Amplicon” means the product of a polynucleotide amplification reaction. That is, it is a population of polynucleotides, usually double stranded, that are replicated from one or more starting sequences. The one or more starting sequences may be one or more copies of the same sequence, or it may be a mixture of different sequences. Amplicons may be produced by a variety of amplification reactions whose products are multiple replicates of one or more target nucleic acids. Generally, amplification reactions producing amplicons are “template-driven” in that base pairing of reactants, either nucleotides or oligonucleotides, have complements in a template polynucleotide that are required for the creation of reaction products. In one aspect, template-driven reactions are primer extensions with a nucleic acid polymerase or oligonucleotide ligations with a nucleic acid ligase. Such reactions include, but are not limited to, polymerase chain reactions (PCRs), linear polymerase reactions, ligase chain reactions (LCRs), strand-displacement reactions (SDAs), nucleic acid sequence-based amplification (NASBAs), rolling circle amplifications, and the like, disclosed in the following references that are incorporated herein by reference: Mullis et al, U.S. Pat. Nos. 4,683,195; 4,965,188; 4,683,202; 4,800,159 (PCR); Gelfand et al, U.S. Pat. No. 5,210,015 (real-time PCR with “taqman” probes); Wittwer et al, U.S. Pat. No. 6,174,670; Landegren et al, U.S. Pat. No. 4,988,617 (“LCR”); Birkenmeyer et al, U.S. Pat. No. 5,427,930 (“gap-LCR”); Kacian et al, U.S. Pat. No. 5,399,491 (“NASBA”); Walker, U.S. Pat. Nos. 5,648,211; 5,712,124 (“SDA”); Lizardi, U.S. Pat. No. 5,854,033; Aono et al, Japanese patent publ. JP 4-262799 (rolling circle amplification); and the like. In one aspect, amplicons of the invention are produced by PCRs. An amplification reaction may be a “real-time” amplification if a detection chemistry is available that permits a reaction product to be measured as the amplification reaction progresses, e.g. “real-time PCR” described below, or “real-time NASBA” as described in Leone et al, Nucleic Acids Research, 26: 2150-2155 (1998), and like references. As used herein, the term “amplifying” means performing an amplification reaction. A “reaction mixture” means a solution containing all the necessary reactants for performing a reaction, which may include, but not be limited to, buffering agents to maintain pH at a selected level during a reaction, salts, co-factors, scavengers, and the like.

“BAD protein” means a human protein capable of forming a stable complex with a human 14-3-3 protein whenever phosphorylated at Ser-112 and/or Ser-155, and capable of forming a stable complex with a human Bcl-2 protein whenever there is no phosphate group attached to Ser-112 and Ser-155, such BAD protein having an amino acid sequence substantially identical to that described under NCBI accession number AAH01901 or in Strausberg et al, Proc. Natl. Acad. Sci., 99: 16899-16903 (2002). In one aspect, a BAD protein hereunder is at least eighty percent identical, and more preferably ninety percent identical, to the amino acid described under NCBI accession number AAH01901 or in Strausberg et al, Proc. Natl. Acad. Sci., 99: 16899-16903 (2002).

“Bcl-2 protein” means a human protein capable of forming a stable complex with a human BAD protein whenever the BAD protein has no phosphate group attached to Ser-112 or Ser-155, and having an amino acid sequence substantially identical to that described under NCBI accession number AAH17214 or in Strausberg et al, Proc. Natl. Acad. Sci., 99: 16899-16903 (2002). In one aspect, a Bcl-2 protein hereunder is at least eighty percent identical, and more preferably ninety percent identical, to the amino acid described under NCBI accession number AAH17214 or in Strausberg et al, Proc. Natl. Acad. Sci., 99: 16899-16903 (2002).

“BCI-XL protein” means a human protein capable of forming a stable complex with a human BAD protein whenever the BAD protein has no phosphate group attached to Ser-112 or Ser-155, and having an amino acid sequence substantially identical to that described under NCBI accession number NP_{—}620120 or in Oltvai et al, Cell, 74: 609-619 (1993). In one aspect, a Bcl-X_{L} protein hereunder is at least eighty percent identical, and more preferably ninety percent identical, to the amino acid described under NCBI accession number NP_{—}620120 or in Oltvai et al, Cell, 74: 609-619 (1993).

“BH3 only protein” means a human protein containing a BH3 domain, but not a BH1, BH2, or BH4 domains, and is capable of forming a stable complex with a Bcl-2 protein. Polypeptide domains, BH1, BH2, BH3, and BH4, are characteristic domains of the Bcl-2 family of proteins, which are described in the following references; Baell and Huang, Biochem. Pharmacology, 64: 851-863 (2002); Sattler et al, Science, 275: 983-985 (1997); Gross et al, Genes & Development, 13: 1899-1911 (1999); and Puthalakath et al, Cell Death and Differentiation, 9: 505-512 (2002); which are incorporated by reference. A BH3 domain is represented by the following consensus sequence of 12 amino acids: “-I-A-X₁—X₂-L-R—R—I-G-D-E-F-,” wherein X₁ is any amino acid and X₂ is a charged amino acid. Preferably, a BH3 domain comprises an amino acid sequence of the consensus sequence, or a sequence having from 1 to 4 conservative amino acid substitutions and/or deletions with respect to the consensus sequence, with the proviso that the leucine (L) in position five and the aspartic acid (D) in position 10 are not substituted or deleted. Exemplary BH3 only proteins include BID, BAD, BIK, BLK, HRK, BIM, NIP3, and NIX/BNIP3, descriptions of which are available from the National Center for Biotechnology Information (NCBI). A preferred BH3 only protein is BAD.

“Antibody” means an immunoglobulin that specifically binds to, and is thereby defined as complementary with, a particular spatial and polar organization of another molecule. The antibody can be monoclonal or polyclonal and can be prepared by techniques that are well known in the art such as immunization of a host and collection of sera (polyclonal) or by preparing continuous hybrid cell lines and collecting the secreted protein (monoclonal), or by cloning and expressing nucleotide sequences or mutagenized versions thereof coding at least for the amino acid sequences required for specific binding of natural antibodies. Antibodies may include a complete immunoglobulin or fragment thereof, which immunoglobulins include the various classes and isotypes, such as IgA, IgD, IgE, IgG1, IgG2a, IgG2b and IgG3, IgM, etc. Fragments thereof may include Fab, Fv and F(ab′)2, Fab′, and the like. In addition, aggregates, polymers, and conjugates of immunoglobulins or their fragments can be used where appropriate so long as binding affinity for a particular polypeptide is maintained. Guidance in the production and selection of antibodies for use in immunoassays, including such assays employing releasable signaling polynucleotide (as described below) can be found in readily available texts and manuals, e.g. Harlow and Lane, Antibodies: A Laboratory Manual (Cold Spring Harbor Laboratory Press, New York, 1988); Howard and Bethell, Basic Methods in Antibody Production and Characterization (CRC Press, 2001); Wild, editor, The Immunoassay Handbook (Stockton Press, New York, 1994), and the like.

“Antibody binding composition” means a molecule or a complex of molecules that comprises one or more antibodies, or fragments thereof, and derives its binding specificity from such antibody or antibody fragment. Antibody binding compositions include, but are not limited to, (i) antibody pairs in which a first antibody binds specifically to a target molecule and a second antibody binds specifically to a constant region of the first antibody; a biotinylated antibody that binds specifically to a target molecule and a streptavidin protein, which protein is derivatized with moieties such as signaling polynucleotides or photosensitizers, or the like, via a biotin moiety; (ii) antibodies specific for a target molecule and conjugated to a polymer, such as dextran, which, in turn, is derivatized with moieties such as signaling polynucleotides or photosensitizers, either directly by covalent bonds or indirectly via streptavidin-biotin linkages; (iii) antibodies specific for a target molecule and conjugated to a bead, or microbead, or other solid phase support, which, in turn, is derivatized either directly or indirectly with moieties such as signaling polynucleotides or photosensitizers, or polymers containing the latter.

“Antigenic determinant,” or “epitope” means a site on the surface of a molecule, usually a protein, to which a single antibody molecule binds; generally a protein has several or many different antigenic determinants and reacts with antibodies of many different specificities. A preferred antigenic determinant is a phosphorylation site of a protein.

“Binding moiety” means any molecule to which signaling polynucleotides can be directly or indirectly attached that is capable of specifically binding to an analyte. Binding moieties include, but are not limited to, antibodies, antibody binding compositions, peptides, proteins, nucleic acids, and organic molecules having a molecular weight of up to 1000 daltons and consisting of atoms selected from the group consisting of hydrogen, carbon, oxygen, nitrogen, sulfur, and phosphorus. Preferably, binding moieties are antibodies or antibody binding compositions.

“Cancer” and “cancerous” refer to or describe the physiological condition in mammals that is typically characterized by unregulated cell growth. Examples of cancer include, but are not limited to, carcinoma, lymphoma, blastoma, sarcoma, and leukemia. More particular examples of such cancers include squamous cell cancer, small-cell lung cancer, non-small cell lung cancer, gastrointestinal cancer, pancreatic cancer, glioblastoma, cervical cancer, ovarian cancer, liver cancer, bladder cancer, hepatoma, breast cancer, colon cancer, colorectal cancer, endometrial carcinoma, salivary gland carcinoma, kidney cancer, prostate cancer, vulval cancer, thyroid cancer, hepatic carcinoma and various types of head and neck cancer.

“Complementary or substantially complementary” refers to the hybridization or base pairing or the formation of a duplex between nucleotides or nucleic acids, such as, for instance, between the two strands of a double stranded DNA molecule or between an oligonucleotide primer and a primer binding site on a single stranded nucleic acid. Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

“Complex” as used herein means an assemblage or aggregate of molecules in direct or indirect contact with one another. In one aspect, “contact,” or more particularly, “direct contact” in reference to a complex of molecules, or in reference to specificity or specific binding, means two or more molecules are close enough so that attractive noncovalent interactions, such as Van der Waal forces, hydrogen bonding, ionic and hydrophobic interactions, and the like, dominate the interaction of the molecules. In such an aspect, a complex of molecules is stable in that under assay conditions the complex is thermodynamically more favorable than a non-aggregated, or non-complexed, state of its component molecules. As used herein, “complex” usually refers to a stable aggregate of two or more proteins, and is equivalently referred to as a “protein-protein complex.” Most typically, a “complex” refers to a stable aggregate of two proteins.

“Dimer” in reference to cell surface membrane receptors means a complex of two or more membrane-bound receptor proteins that may be the same or different. Dimers of identical receptors are referred to as “homodimers” and dimers of different receptors are referred to as “heterodimers.” Dimers usually consist of two receptors in contact with one another. Dimers may be created in a cell surface membrane by passive processes, such as Van der Waal interactions, and the like, as described above in the definition of “complex,” or dimers may be created by active processes, such as by ligand-induced dimerization, covalent linkages, interaction with intracellular components, or the like, e.g. Schlessinger, Cell, 103: 211-225 (2000). As used herein, the term “dimer” is understood to refer to “cell surface membrane receptor dimer,” unless understood otherwise from the context.

“Duplex” means at least two oligonucleotides and/or polynucleotides that are fully or partially complementary undergo Watson-Crick type base pairing among all or most of their nucleotides so that a stable complex is formed. The terms “annealing” and “hybridization” are used interchangeably to mean the formation of a stable duplex. “Perfectly matched” in reference to a duplex means that the poly- or oligonucleotide strands making up the duplex form a double stranded structure with one another such that every nucleotide in each strand undergoes Watson-Crick base pairing with a nucleotide in the other strand. The term “duplex” comprehends the pairing of nucleoside analogs, such as deoxyinosine, nucleosides with 2-aminopurine bases, PNAs, and the like, that may be employed. A “mismatch” in a duplex between two oligonucleotides or polynucleotides means that a pair of nucleotides in the duplex fails to undergo Watson-Crick bonding.

“Disease status” includes, but is not limited to, the following features: likelihood of contracting a disease, presence or absence of a disease, prognosis of disease severity, and likelihood that a patient will respond to treatment by a particular therapeutic agent that acts through a receptor complex. In regard to cancer, “disease status” further includes detection of precancerous or cancerous cells or tissues, the selection of patients that are likely to respond to treatment by a therapeutic agent that acts through one or more receptor complexes, such as one or more receptor dimers, and the ameliorative effects of treatment with such therapeutic agents. In one aspect, disease status in reference to Her receptor complexes means likelihood that a cancer patient will respond to treatment by a Her dimer-acting drug. Preferably, such cancer patient is a breast or ovarian cancer patient and such Her dimer-acting drugs include Omnitarg™ (2C4), Herceptin, ZD-1839 (Iressa), and OSI-774 (Tarceva). In another aspect, disease status in reference to PDGFR receptor complexes means the likelihood that a patient suffering from a disease characterized by inappropriate fibrosis will respond to treatment by a PDGFR dimer-acting drug. Preferably, such disease includes cancer, and kidney fibrosis. In another aspect, disease status in reference to VEGF receptor complexes means the likelihood that a patient suffering from a disease characterized by inappropriate angiogenesis, such as solid tumors, will respond to treatment by a VEGF dimer-acting drug.

“ErbB receptor” or “Her receptor” is a receptor protein tyrosine kinase which belongs to the ErbB receptor family and includes EGFR (“Her1”), ErbB2 (“Her2”), ErbB3 (“Her3”) and ErbB4 (“Her4”) receptors. The ErbB receptor generally comprises an extracellular domain, which may bind an ErbB ligand; a lipophilic transmembrane domain; a conserved intracellular tyrosine kinase domain; and a carboxyl-terminal signaling domain harboring several tyrosine residues which can be phosphorylated. The ErbB receptor may be a native sequence ErbB receptor or an amino acid sequence variant thereof. Preferably the ErbB receptor is native sequence human ErbB receptor.

The terms “ErbB1”, “epidermal growth factor receptor” and “EGFR” and “Her1” are used interchangeably herein and refer to native sequence EGFR as disclosed, for example, in Carpenter et al. Ann. Rev. Biochem. 56:881-914 (1987), including variants thereof (e.g. a deletion mutant EGFR as in Humphrey et al. PNAS (USA) 87:4207-4211 (1990)). erbB1 refers to the gene encoding the EGFR protein product. Examples of antibodies which bind to EGFR include MAb 579 (ATCC CRL RB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.).

“Her2”, “ErbB2” “c-Erb-B2” are used interchangeably. Unless indicated otherwise, the terms “ErbB2” “c-Erb-B2” and “Her2” when used herein refer to the human protein. The human ErbB2 gene and ErbB2 protein are, for example, described in Semba et al., PNAS (USA) 82:6497-650 (1985) and Yamamoto et al. Nature 319:230-234 (1986) (Genebank accession number X03363). Examples of antibodies that specifically bind to Her2 are disclosed in U.S. Pat. Nos. 5,677,171; 5,772,997; Fendly et al, Cancer Res., 50: 1550-1558 (1990); and the like.

“ErbB3” and “Her3” refer to the receptor polypeptide as disclosed, for example, in U.S. Pat. Nos. 5,183,884 and 5,480,968 as well as Kraus et al. PNAS (USA) 86:9193-9197 (1989), including variants thereof. Examples of antibodies which bind Her3 are described in U.S. Pat. No. 5,968,511, e.g. the 8B8 antibody (ATCC HB 12070).

The terms “ErbB4” and “Her4” herein refer to the receptor polypeptide as disclosed, for example, in Plowman et al., Proc. Natl. Acad. Sci. USA, 90:1746-1750 (1993); and Plowman et al., Nature, 366:473-475 (1993). Antibodies to Her4 are disclosed in U.S. patent SSSSSSSSS

“Insulin-like growth factor-1 receptor” or “1GF-1R” means a human receptor tyrosine kinase substantially identical to those disclosed in Ullrich et al, EMBO J., 5: 2503-2512 (1986) or Steele-Perkins et al, J. Biol. Chem., 263: 11486-11492 (1988).

“Fluorescent indicator” means a probe that is capable of generating a fluorescent signal in the presence of a product of an amplification reaction (i.e. an “amplification product ”) such that as product accumulates in the reaction mixture the signal of the fluorescent indicator increases, at least over a predetermined range of concentrations. Fluorescent indicators may be non-specific, such as intercalating dyes that bind to double stranded DNA products, e.g. YO-PRO-1, SYBR green 1, and the like, Ishiguro et al, Anal. Biochem., 229: 207-213 (1995); Tseng et al, Anal. Biochem., 245: 207-212 (1997); Morrison et al, Biotechniques, 24: 954-962 (1998); or such as primers having hairpin structures with a fluorescent molecule held in proximity to a fluorescent quencher until forced apart by primer extension, e.g. Whitecombe et al, Nature Biotechnology, 17: 804-807 (1999)(“Amplifluor™ primers”). Fluorescent indicators also may be target sequence specific, usually comprising a fluorescent molecule in proximity to a fluorescent quencher until an oligonucleotide moiety to which they are attached specifically binds to an amplification product, e.g. Gelfand et al, U.S. Pat. No. 5,210,015 (“taqman”); Nazarenko et al, Nucleic Acids Research, 25: 2516-2521 (1997)(“scorpion probes”); Tyagi et al, Nature Biotechnology, 16: 49-53 (1998)(“molecular beacons”). Fluorescent indicators may be used in connection with real-time PCR, or they may be used to measure the total amount of reaction product at the completion of a reaction.

“Internal standard” means a nucleic acid sequence that is amplified in the same amplification reaction as a target polynucleotide in order to permit absolute or relative quantification of the target polynucleotide in a sample. An internal standard may be endogenous or exogenous. That is, an internal standard may occur naturally in the sample, or it may be added to the sample prior to amplification. In one aspect, multiple exogenous internal standard sequences may be added to a reaction mixture in a series of predetermined concentrations to provide a calibration to which a target amplicon may be compared to determine the quantity of its corresponding target polynucleotide in a sample. Selection of the number, sequences, lengths, and other characteristics of exogenous internal standards is a routine design choice for one of ordinary skill in the art. Preferably, endogenous internal standards, also referred to herein as “reference sequences,” are sequences natural to a sample that correspond to minimally regulated genes that exhibit a constant and cell cycle-independent level of transcription, e.g. Selvey et al, Mol. Cell Probes, 15: 307-311 (2001). Exemplary reference sequences include, but are not limited to, sequences from the following genes: GAPDH, β₂-microglobulin, 18S ribosomal RNA, and β-actin (although see Selvey et al, cited above).

“I-kB protein,” or “inhibitor of NF-kB protein,” means a human protein capable of forming a stable complex with a human NF-kB protein whenever the I-kB protein is in a partially phosphorylated state. In one aspect, an I-kB protein has an amino acid sequence substantially identical to that described under NCBI accession number 000221 or in Baeuerle and Baltimore, Science, 242: 540-546 (1988).

“Isolated” in reference to a polypeptide or protein means substantially separated from the components of its natural environment. Preferably, an isolated polypeptide or protein is a composition that consists of at least eighty percent of the polypeptide or protein identified by sequence on a weight basis as compared to components of its natural environment; more preferably, such composition consists of at least ninety-five percent of the polypeptide or protein identified by sequence on a weight basis as compared to components of its natural environment; and still more preferably, such composition consists of at least ninety-nine percent of the polypeptide or protein identified by sequence on a weight basis as compared to components of its natural environment. Most preferably, an isolated polypeptide or protein is a homogeneous composition that can be resolved as a single spot after conventional separation by two-dimensional gel electrophoresis based on molecular weight and isoelectric point. Protocols for such analysis by conventional two-dimensional gel electrophoresis are well known to one of ordinary skill in the art, e.g. Hames and Rickwood, Editors, Gel Electrophoresis of Proteins: A Practical Approach (IRL Press, Oxford, 1981); Scopes, Protein Purification (Springer-Verlag, New York, 1982); Rabilloud, Editor, Proteome Research: Two-Dimensional Gel Electrophoresis and Identification Methods (Springer-Verlag, Berlin, 2000).

“Kit” refers to any delivery system for delivering materials or reagents for carrying out a method of the invention. In the context of reaction assays, such delivery systems include systems that allow for the storage, transport, or delivery of reaction reagents (e.g., probes, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. Such contents may be delivered to the intended recipient together or separately. For example, a first container may contain an enzyme for use in an assay, while a second container contains probes.

“Microarray” refers to a solid phase support having a planar surface, which carries an array of nucleic acids, each member of the array comprising identical copies of an oligonucleotide or polynucleotide immobilized to a spatially defined region or site, which does not overlap with those of other members of the array; that is, the regions or sites are spatially discrete. Spatially defined hybridization sites may additionally be “addressable” in that its location and the identity of its immobilized oligonucleotide are known or predetermined, for example, prior to its use. Typically, the oligonucleotides or polynucleotides are single stranded and are covalently attached to the solid phase support. The density of non-overlapping regions containing nucleic acids in a microarray is typically greater than 100 per cm², and more preferably, greater than 1000 per cm². Microarray technology is reviewed in the following references: Schena, Editor, Microarrays: A Practical Approach (IRL Press, Oxford, 2000); Southern, Current Opin. Chem. Biol., 2: 404-410 (1998); Nature Genetics Supplement, 21: 1-60 (1999). As used herein, “random microarray” refers to a microarray whose spatially discrete regions of oligonucleotides or polynucleotides are not spatially addressed. That is, the identity of the attached oligonucleoties or polynucleotides is not discernable, at least initially, from its location. Preferably, random microarrays are planar arrays of microbeads wherein each microbead has attached a single kind of hybridization tag complement, such as from a minimally cross-hybridizing set of oligonucleotides. Arrays of microbeads may be formed in a variety of ways, e.g. Brenner et al, Nature Biotechnology, 18: 630-634 (2000); Tulley et al, U.S. Pat. No. 6,133,043; Stuelpnagel et al, U.S. Pat. No. 6,396,995; Chee et al, U.S. Pat. No. 6,544,732; and the like. Likewise, after formation, microbeads, or oligonucleotides thereof, in a random array may be identified in a variety of ways, including by optical labels, e.g. fluorescent dye ratios or quantum dots, shape, sequence analysis, or the like.

“NF-kB protein,” or “nuclear factor kappa B protein,” means a human protein capable of forming a stable complex with a human I-kB protein whenever the I-kB protein is in a partially phosphorylated state, and having an amino acid sequence substantially identical to that described under NCBI accession number NP_{—}003989. In one aspect, an NF-kB protein hereunder is at least eighty percent identical, and more preferably ninety percent identical, to the amino acid described under NCBI accession number NP_{—}003989.

“Percent identical,” or like term, used in respect of the comparison of a reference sequence and another sequence (i.e. a “candidate” sequence) means that in an optimal alignment between the two sequences, the candidate sequence is identical to the reference sequence in a number of subunit positions equivalent to the indicated percentage, the subunits being nucleotides for polynucleotide comparisons or amino acids for polypeptide comparisons. As used herein, an “optimal alignment” of sequences being compared is one that maximizes matches between subunits and minimizes the number of gaps employed in constructing an alignment. Percent identities may be determined with commercially available implementations of algorithms described by Needleman and Wunsch, J. Mol. Biol., 48: 443-453 (1970)(“GAP” program of Wisconsin Sequence Analysis Package, Genetics Computer Group, Madison, Wis.). Other software packages in the art for constructing alignments and calculating percentage identity or other measures of similarity include the “BestFit” program, based on the algorithm of Smith and Waterman, Advances in Applied Mathematics, 2: 482-489 (1981) (Wisconsin Sequence Analysis Package, Genetics Computer Group, Madison, Wis.). In other words, for example, to obtain a polypeptide having an amino acid sequence at least 95 percent identical to a reference amino acid sequence, up to five percent of the amino acid residues in the reference sequence many be deleted or substituted with another amino acid, or a number of amino acids up to five percent of the total amino acid residues in the reference sequence may be inserted into the reference sequence. These alterations of the reference sequence many occur at the amino or carboxy terminal positions of the reference amino acid sequence or anywhere between those terminal positions, interspersed either individually among residues in the reference sequence of in one or more contiguous groups with in the references sequence. It is understood that in making comparisons with reference sequences of the invention that candidate sequence may be a component or segment of a larger polypeptide or polynucleotide and that such comparisons for the purpose computing percentage identity is to be carried out with respect to the relevant component or segment.

“Phosphatidylinositol 3 kinase protein,” or equivalently a “PI3K protein,” means a human intracellular protein of the set of human proteins describe under NCBI accession numbers NP_{—}852664, NP_{—}852556, and NP_{—}852665, and proteins having amino acid sequences substantially identical thereto.

“Platelet-derived growth factor receptor” or “PDGFR” means a human receptor tyrosine kinase protein that is substantially identical to PDGFRα or PDGFRβ, or variants thereof, described in Heldin et al, Physiological Reviews, 79: 1283-1316 (1999). In one aspect, the invention includes determining the status of cancers, pre-cancerous conditions, fibrotic or sclerotic conditions by measuring one or more dimers of the following group: PDGFRα homodimers, PDGFRβ homodimers, and PDGFRα-PDGFRβ heterodimers. In particular, fibrotic conditions include lung or kidney fibrosis, and sclerotic conditions include atherosclerosis. Cancers include, but are not limited to, breast cancer, colorectal carcinoma, glioblastoma, and ovarian carcinoma. Reference to “PDGFR” alone is understood to mean “PDGFRα” or “PDGFRβ.”

“Polymerase chain reaction,” or “PCR,” means a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors well-known to those of ordinary skill in the art, e.g. exemplified by the references: McPherson et al, editors, PCR: A Practical Approach and PCR2: A Practical Approach (IRL Press, Oxford, 1991 and 1995, respectively). For example, in a conventional PCR using Taq DNA polymerase, a double stranded target nucleic acid may be denatured at a temperature >90° C., primers annealed at a temperature in the range 50-75° C., and primers extended at a temperature in the range 72-78° C. The term “PCR” encompasses derivative forms of the reaction, including but not limited to, RT-PCR, real-time PCR, nested PCR, quantitative PCR, multiplexed PCR, and the like. Reaction volumes range from a few hundred nanoliters, e.g. 200 nL, to a few hundred μL, e.g. 200 μL. “Reverse transcription PCR,” or “RT-PCR,” means a PCR that is preceded by a reverse transcription reaction that converts a target RNA to a complementary single stranded DNA, which is then amplified, e.g. Tecott et al, U.S. Pat. No. 5,168,038, which patent is incorporated herein by reference. “Real-time PCR” means a PCR for which the amount of reaction product, i.e. amplicon, is monitored as the reaction proceeds. There are many forms of real-time PCR that differ mainly in the detection chemistries used for monitoring the reaction product, e.g. Gelfand et al, U.S. Pat. No. 5,210,015 (“taqman”); Wittwer et al, U.S. Pat. Nos. 6,174,670 and 6,569,627 (intercalating dyes); Tyagi et al, U.S. Pat. No. 5,925,517 (molecular beacons); which patents are incorporated herein by reference. Detection chemistries for real-time PCR are reviewed in Mackay et al, Nucleic Acids Research, 30: 1292-1305 (2002), which is also incorporated herein by reference. “Nested PCR” means a two-stage PCR wherein the amplicon of a first PCR becomes the sample for a second PCR using a new set of primers, at least one of which binds to an interior location of the first amplicon. As used herein, “initial primers” in reference to a nested amplification reaction mean the primers used to generate a first amplicon, and “secondary primers” mean the one or more primers used to generate a second, or nested, amplicon. “Multiplexed PCR” means a PCR wherein multiple target sequences (or a single target sequence and one or more reference sequences) are simultaneously carried out in the same reaction mixture, e.g. Bernard et al, Anal. Biochem., 273: 221-228 (1999)(two-color real-time PCR). Usually, distinct sets of primers are employed for each sequence being amplified. Typically, the number of target sequences in a multiplex PCR is in the range of from 2 to 10, or from 2 to 6, or more typically, from 2 to 4. “Quantitative PCR” means a PCR designed to measure the abundance of one or more specific target sequences in a sample or specimen. Quantitative PCR includes both absolute quantitation and relative quantitation of such target sequences. Quantitative measurements are made using one or more reference sequences that may be assayed separately or together with a target sequence. The reference sequence may be endogenous or exogenous to a sample or specimen, and in the latter case, may comprise one or more competitor templates. Typical endogenous reference sequences include segments of transcripts of the following genes: β-actin, GAPDH, β₂-microglobulin, ribosomal RNA, and the like. Techniques for quantitative PCR are well-known to those of ordinary skill in the art, as exemplified in the following references that are incorporated by reference: Freeman et al, Biotechniques, 26: 112-126 (1999); Becker-Andre et al, Nucleic Acids Research, 17: 9437-9447 (1989); Zimmerman et al, Biotechniques, 21: 268-279 (1996); Diviacco et al, Gene, 122: 3013-3020 (1992); Becker-Andre et al, Nucleic Acids Research, 17: 9437-9446 (1989); and the like.

“Polynucleotide” and “oligonucleotide” are used interchangeably and each means a linear polymer of nucleotide monomers. Monomers making up polynucleotides and oligonucleotides are capable of specifically binding to a natural polynucleotide by way of a regular pattern of monomer-to-monomer interactions, such as Watson-Crick type of base pairing, base stacking, Hoogsteen or reverse Hoogsteen types of base pairing, or the like. Such monomers and their internucleosidic linkages may be naturally occurring or may be analogs thereof, e.g. naturally occurring or non-naturally occurring analogs. Non-naturally occurring analogs may include PNAs, phosphorothioate internucleosidic linkages, bases containing linking groups permitting the attachment of labels, such as fluorophores, or haptens, and the like. Whenever the use of an oligonucleotide or polynucleotide requires enzymatic processing, such as extension by a polymerase, ligation by a ligase, or the like, one of ordinary skill would understand that oligonucleotides or polynucleotides in those instances would not contain certain analogs of intemucleosidic linkages, sugar moieties, or bases at any or some positions. Polynucleotides typically range in size from a few monomeric units, e.g. 5-40, when they are usually referred to as “oligonucleotides,” to several thousand monomeric units. Whenever a polynucleotide or oligonucleotide is represented by a sequence of letters (upper or lower case), such as “ATGCCTG,” it will be understood that the nucleotides are in 5′→3′ order from left to right and that “A” denotes deoxyadenosine, “C” denotes deoxycytidine, “G” denotes deoxyguanosine, and “T” denotes thymidine, “I” denotes deoxyinosine, “U” denotes uridine, unless otherwise indicated or obvious from context. Unless otherwise noted the terminology and atom numbering conventions will follow those disclosed in Strachan and Read, Human Molecular Genetics 2 (Wiley-Liss, New York, 1999). Usually polynucleotides comprise the four natural nucleosides (e.g. deoxyadenosine, deoxycytidine, deoxyguanosine, deoxythymidine for DNA or their ribose counterparts for RNA) linked by phosphodiester linkages; however, they may also comprise non-natural nucleotide analogs, e.g. including modified bases, sugars, or intemucleosidic linkages. It is clear to those skilled in the art that where an enzyme has specific oligonucleotide or polynucleotide substrate requirements for activity, e.g. single stranded DNA, RNA/DNA duplex, or the like, then selection of appropriate composition for the oligonucleotide or polynucleotide substrates is well within the knowledge of one of ordinary skill, especially with guidance from treatises, such as Sambrook et al, Molecular Cloning, Second Edition (Cold Spring Harbor Laboratory, New York, 1989), and like references.

“Primer” means an oligonucleotide, either natural or synthetic that is capable, upon forming a duplex with a polynucleotide template, of acting as a point of initiation of nucleic acid synthesis and being extended from its 3′ end along the template so that an extended duplex is formed. Extension of a primer is usually carried out with a nucleic acid polymerase, such as a DNA or RNA polymerase. The sequence of nucleotides added in the extension process is determined by the sequence of the template polynucleotide. Usually primers are extended by a DNA polymerase. Primers usually have a length in the range of from 14 to 40 nucleotides, or in the range of from 18 to 36 nucleotides. Primers are employed in a variety of nucleic amplification reactions, for example, linear amplification reactions using a single primer, or polymerase chain reactions, employing two or more primers. Guidance for selecting the lengths and sequences of primers for particular applications is well known to those of ordinary skill in the art, as evidenced by the following references that are incorporated by reference: Dieffenbach, editor, PCR Primer: A Laboratory Manual, 2^{nd} Edition (Cold Spring Harbor Press, New York, 2003).

“Readout” means a parameter, or parameters, which are measured and/or detected that can be converted to a number or value. In some contexts, readout may refer to an actual numerical representation of such collected or recorded data. For example, a readout of fluorescent intensity signals from a microarray is the address and fluorescence intensity of a signal being generated at each hybridization site of the microarray; thus, such a readout may be registered or stored in various ways, for example, as an image of the microarray, as a table of numbers, or the like.

“Polypeptide” refers to a class of compounds composed of amino acid residues chemically bonded together by amide linkages with elimination of water between the carboxy group of one amino acid and the amino group of another amino acid. A polypeptide is a polymer of amino acid residues, which may contain a large number of such residues. Peptides are similar to polypeptides, except that, generally, they are comprised of a lesser number of amino acids. Peptides are sometimes referred to as oligopeptides. There is no clear-cut distinction between polypeptides and peptides. For convenience, in this disclosure and claims, the term “polypeptide” will be used to refer generally to peptides and polypeptides. The amino acid residues may be natural or synthetic.

“Protein” refers to a polypeptide, usually synthesized by a biological cell, folded into a defined three-dimensional structure. Proteins are generally from about 5,000 to about 5,000,000 or more in molecular weight, more usually from about 5,000 to about 1,000,000 molecular weight, and may include posttranslational modifications, such acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, farnesylation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, phosphorylation, prenylation, racemization, selenoylation, sulfation, and ubiquitination, e.g. Wold, F., Post-translational Protein Modifications: Perspectives and Prospects, pgs. 1-12 in Post-translational Covalent Modification of Proteins, B. C. Johnson, Ed., Academic Press, New York, 1983. Proteins include, by way of illustration and not limitation, cytokines or interleukins, enzymes such as, e.g., kinases, proteases, galactosidases and so forth, protamines, histones, albumins, immunoglobulins, scleroproteins, phosphoproteins, mucoproteins, chromoproteins, lipoproteins, nucleoproteins, glycoproteins, T-cell receptors, proteoglycans, and the like.

“Receptor tyrosine kinase,” or “RTK,” means a human receptor protein having intracellular kinase activity and being selected from the RTK family of proteins described in Schlessinger, Cell, 103: 211-225 (2000); and Blume-Jensen and Hunter (cited above). “Receptor tyrosine kinase dimer” means a complex in a cell surface membrane comprising two receptor tyrosine kinase proteins. In some aspects, a receptor tyrosine kinase dimer may comprise two covalently linked receptor tyrosine kinase proteins. Exemplary RTK dimers are listed below. RTK dimers of particular interest are Her receptor dimers and VEGFR dimers.

“Reference sample” means one or more cell or tissue samples that are representative of a normal or non-diseased state to which measurements on patient samples are compared to determine whether a receptor complex is present in excess or is present in reduced amount in the patient sample. The nature of the reference sample is a matter of design choice for a particular assay and may be derived or determined from normal tissue of the patient him- or herself, or from tissues from a population of healthy individuals. Preferably, values relating to amounts of receptor complexes on reference samples are obtained under essentially identical experimental conditions as corresponding values for patient samples being tested. Reference samples may be from the same kind of tissue as that the patient sample, or it may be from different tissue types, and the population from which reference samples are obtained may be selected for characteristics that match those of the patient, such as age, sex, race, and the like. Typically, in assays of the invention, amounts of receptor complexes on patient samples are compared to corresponding values of reference samples that have been previously tabulated and are provided as average ranges, average values with standard deviations, or like representations.

“Receptor complex” means a complex that comprises a dimer of cell surface membrane receptors. Receptor complexes may include one or more intracellular proteins, such as adaptor proteins, that form links in the various signaling pathways. Exemplary intracellular proteins that may be part of a receptor complex includes, but is not limit to, PI3K proteins, Grb2 proteins, Grb7 proteins, Shc proteins, and Sos proteins, Src proteins, Cbl proteins, PLCγ proteins, Shp2 proteins, GAP proteins, Nck proteins, Vav proteins, and Crk proteins.

“Sample” or “tissue sample” or “patient sample” or “patient cell or tissue sample” or “specimen” each means a collection of similar cells obtained from a tissue of a subject or patient. The source of the tissue sample may be solid tissue as from a fresh, frozen and/or preserved organ or tissue sample or biopsy or aspirate; blood or any blood constituents; bodily fluids such as cerebral spinal fluid, amniotic fluid, peritoneal fluid, or interstitial fluid; or cells from any time in gestation or development of the subject. The tissue sample may contain compounds which are not naturally intermixed with the tissue in nature such as preservatives, anticoagulants, buffers, fixatives, nutrients, antibiotics, or the like. In one aspect of the invention, tissue samples or patient samples are fixed, particularly conventional formalin-fixed paraffin-embedded samples. Such samples are typically used in an assay for receptor complexes in the form of thin sections, e.g. 3-10 μm thick, of fixed tissue mounted on a microscope slide, or equivalent surface. Such samples also typically undergo a conventional re-hydration procedure, and optionally, an antigen retrieval procedure as a part of, or preliminary to, assay measurements.

“SHC” (standing for “Src homology 2/α-collagen-related”) means any one of a family of adaptor proteins (66, 52, and 46 kDalton) in RTK signaling pathways substantially identical to those described in Pelicci et al, Cell, 70: 93-104 (1992). In one aspect, SHC means the human versions of such adaptor proteins.

“Signaling pathway” or “signal transduction pathway” means a series of molecular events usually beginning with the interaction of cell surface receptor with an extracellular ligand or with the binding of an intracellular molecule to a phosphorylated site of a cell surface receptor that triggers a series of molecular interactions, wherein the series of molecular interactions results in a regulation of gene expression in the nucleus of a cell. “Ras-MAPK pathway” means a signaling pathway that includes the phosphorylation of a MAPK protein subsequent to the formation of a Ras-GTP complex. “PI3K-Akt pathway” means a signaling pathway that includes the phosphorylation of an Akt protein by a PI3K protein.

“Specific” or “specificity” in reference to the binding of one molecule to another molecule, such as a binding compound, or probe, for a target analyte or complex, means the recognition, contact, and formation of a stable complex between the probe and target, together with substantially less recognition, contact, or complex formation of the probe with other molecules. In one aspect, “specific” in reference to the binding of a first molecule to a second molecule means that to the extent the first molecule recognizes and forms a complex with another molecules in a reaction or sample, it forms the largest number of the complexes with the second molecule. In one aspect, this largest number is at least fifty percent of all such complexes form by the first molecule. Generally, molecules involved in a specific binding event have areas on their surfaces or in cavities giving rise to specific recognition between the molecules binding to each other. Examples of specific binding include antibody-antigen interactions, enzyme-substrate interactions, formation of duplexes or triplexes among polynucleotides and/or oligonucleotides, receptor-ligand interactions, and the like.

“Spectrally resolvable” in reference to a plurality of fluorescent labels means that the fluorescent emission bands of the labels are sufficiently distinct, ie. sufficiently non-overlapping, that signaling polynucleotides to which the respective labels are attached can be distinguished on the basis of the fluorescent signal generated by the respective labels by standard photodetection systems, e.g. employing a system of band pass filters and photomultiplier tubes, or the like, as exemplified by the systems described in U.S. Pat. Nos. 4,230,558; 4,811,218, or the like, or in Wheeless et al, pgs. 21-76, in Flow Cytometry: Instrumentation and Data Analysis (Academic Press, New York, 1985).

“Signaling pathway” or “signal transduction pathway” means a series of molecular events usually beginning with the interaction of cell surface receptor with an extracellular ligand or with the binding of an intracellular molecule to a phosphorylated site of a cell surface receptor that results in a regulation of gene expression in the nucleus of a cell. “Ras-MAPK pathway” means a signaling pathway that includes the phosphorylation of a MAPK protein subsequent to the formation of a Ras-GTP complex. “PI3K-Akt pathway” means a signaling pathway that includes the phosphorylation of an Akt protein by a PI3K protein.

“Substantially identical” in reference to proteins or amino acid sequences of proteins in a family of related proteins that are being compared means either that one protein has an amino acid sequence that is at least fifty percent identical to the other protein or that one protein is an isoform or splice variant of the same gene as the other protein. In one aspect, substantially identical means one protein, or amino acid sequence thereof, is at least eighty percent identical to the other protein, or amino acid sequence thereof.

“VEGF receptor” or “VEGFR” as used herein refers to a cellular receptor for vascular endothelial growth factor (VEGF), ordinarily a cell-surface receptor found on vascular endothelial cells, as well as variants thereof which retain the ability to bind human VEGF. VEGF receptors include VEGFR1 (also known as Flt1), VEGFR2 (also know as Flk1 or KDR), and VEGFR3 (also known as Flt4). These receptors are described in DeVries et al., Science 255:989 (1992); Shibuya et al., Oncogene 5:519 (1990); Matthews et al., Proc. Nat. Acad. Sci. 88:9026 (1991); Terman et al., Oncogene 6:1677 (1991); Terman et al., Biochem. Biophys. Res. Commun. 187:1579 (1992). Dimers of VEGF receptors are described in Shibuya, Cell Structure and Function, 26: 25-35 (2001); and Ferrara et al, Nature Medicine, 9: 669-676 (2003); and include VEGFR1 homodimers, VEGFR2 homodimers, VEGFR1-VEGFR2 heterodimers, and VEGFR2-VEGFR3 heterodimers.

### DETAILED DESCRIPTION OF THE INVENTION

The invention provides methods and compositions for detecting or measuring molecular complexes. Generally, the invention employs pairs of compounds, also referred to as “reagent pairs,” comprising (i) one or more binding compounds each cleavably linked to a different, and usually unique, signaling polynucleotide (referred to herein as “signaling reagents”), and (ii) one or more binding compounds each having attached one or more cleaving agents (collectively referred to as a “cleaving probe”). Binding compounds of the invention are usually antibody binding compounds, and especially, monoclonal antibodies. The assays of the invention may be conducted under the same or similar conditions as the immunoassays disclosed in the following references that are incorporated by reference for the teachings of assay conditions: U.S. patent publication 2004/0126818, and PCT publications WO 2004/091384, WO 2004/087887, and WO 2005/019470.

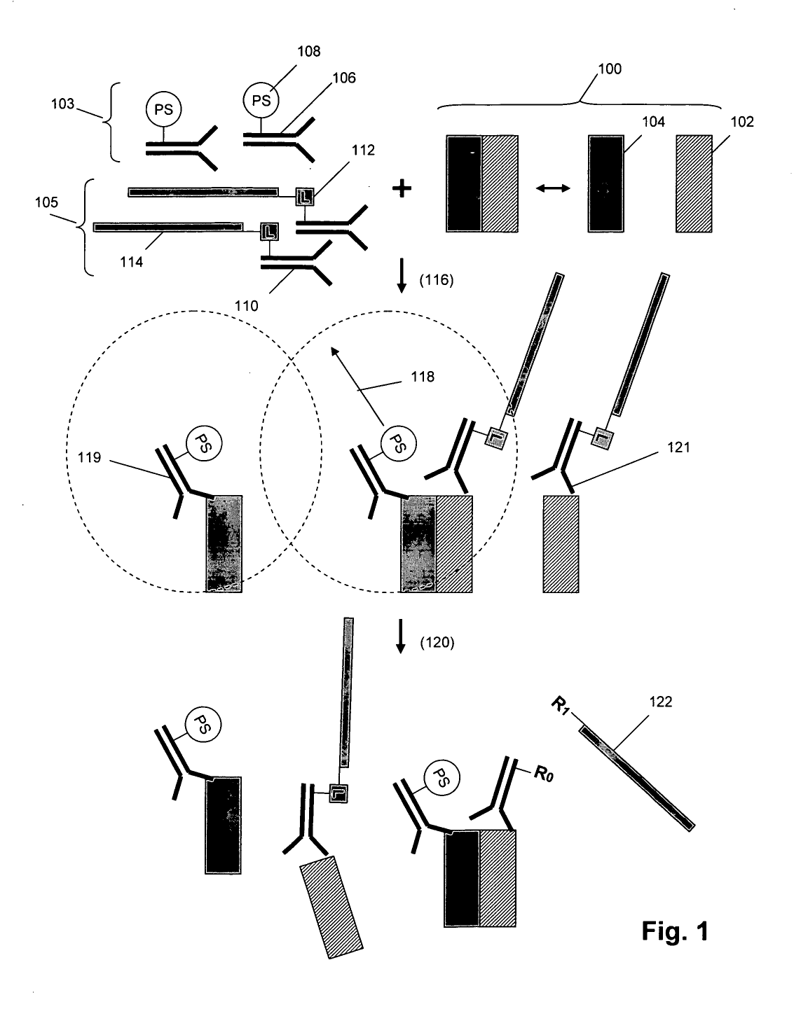

In one aspect, operation of the method of the invention is illustrated in FIG. 1. Cleaving probe (103) comprising antibody binding composition (106) and cleaving agent (108) (such as a photosensitizer, as indicated by the designation “PS”) and signaling reagent (105) comprising antibody binding composition (110) and signaling polynucleotide (114) attached thereto by cleavable linkage (112) are combined with molecular complex (100) (illustrated as a dimer in this case) whose components (102) and (104) are either present in a dimeric state or a monomeric state. Conditions are selected (116) to permit antibody binding compositions (106) and (110) to specifically bind to their target epitopes, after which cleaving agent (108), such as a photosensitizer, like methylene blue, or the like, is induced to generate a reactive species, such as singlet oxygen, that has an effective proximity within which there is a high likelihood of reacting with and cleaving any cleavable linkages (112) to produce (120) a released signaling polynucleotide (122). Reactive species generated by cleaving probes (119) bound to monomers typically do not have cleavable linkages within the effective proximity of its cleaving agent; thus, its activation does not produce (120) released signaling polynucleotides (122). Likewise, cleavable linkages of signaling reagents bound to monomers (121) are outside the effective proximities of any cleaving agents; thus, such configurations do not permit release of signaling polynucleotides. Because different members of a reagent pair bind to different components of a molecular complex, signaling polynucleotides are released only when a molecular complex is present and a cleavable linkage is within an effective proximity of a cleaving agent. Released signaling polynucleotides (122) are then used to detect or quantify molecular complex (100). As mentioned above, in one aspect of the invention, such detection may require isolation or separation of the released signaling polynucleotide followed by amplification, for example, by PCR.

The size and sequence structure of signaling polynucleotides (122) may vary widely and will depend on factors such as whether and what type of amplification scheme is used in a detection step, whether multiple targets being detected by different signaling polynucleotides, i.e. whether an assay is multiplexed, how signaling polynucleotides for different targets are distinguished, and the like. In one aspect, signaling polynucleotides of the invention have lengths in the range of from about 24 to 100 nucleotides, or from about 24 to 64 nucleotides, or from about 24 to 48 nucleotides. In multiplex assays of the invention, multiple signaling polynucleotides may be distinguished in a variety of ways depending on the degree of multiplexing desired. In one aspect, when multiplexing is in the range of five or less, signaling polynucleotides may be distinguished by different fluorescent dyes that may be used to label different primer sequences, or different Taqman probes, or different molecular beacons, or like signal generation means. For higher degrees of multiplexing, a variety of tagging schemes are available, such as use of barcode sequences that are amplified, labeled and hybridized to their complements on an array, such as disclosed by Willis et al, U.S. Pat. No. 6,858,412, or to their complements on a collection of encoded beads, such as disclosed by Chandler et al, U.S. Pat. No. 6,632,526; Chandler et al, U.S. Pat. No. 6,449,562; Weiss et al, U.S. Pat. No. 6,207,392; Bruchez et al, U.S. Pat. No. 6,500,622; and the like, which patents are incorporated by reference. Oligonucleotide tags may also be simply different lengths of DNA inserts that are amplified, labeled and separated for detection, such as disclosed by Mahtani, U.S. Pat. No. 6,221,603; or the like, which patents are incorporated herein by reference.

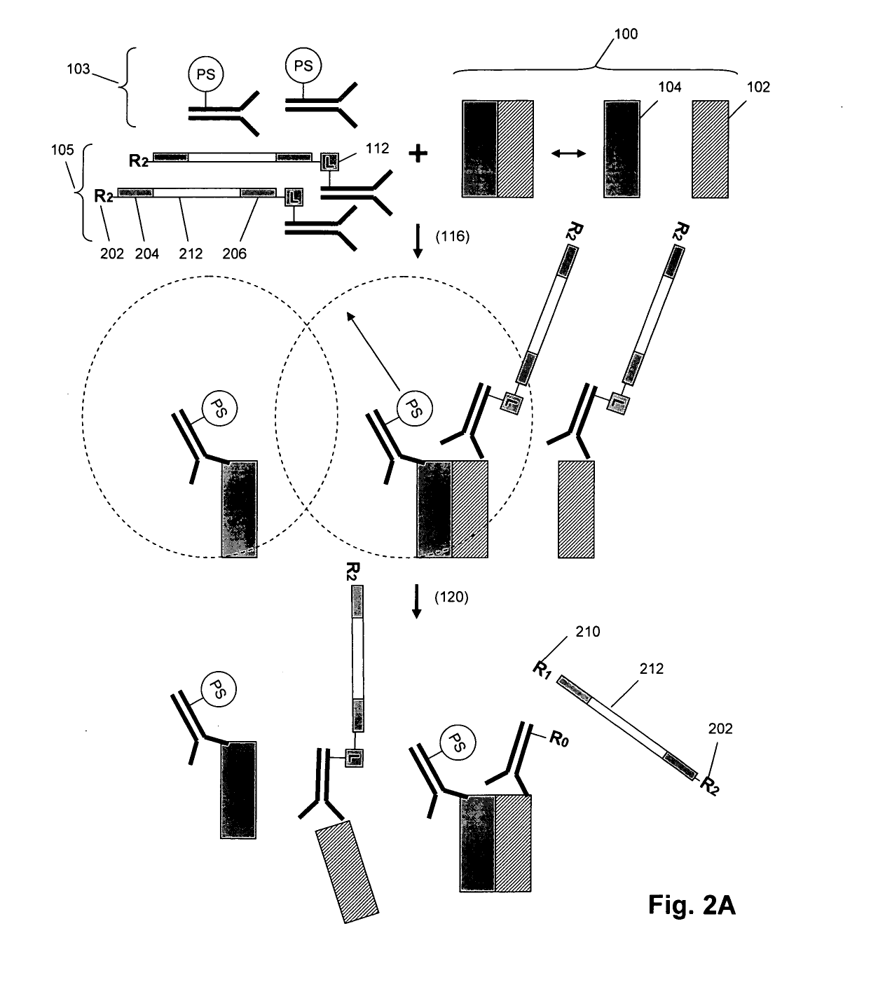

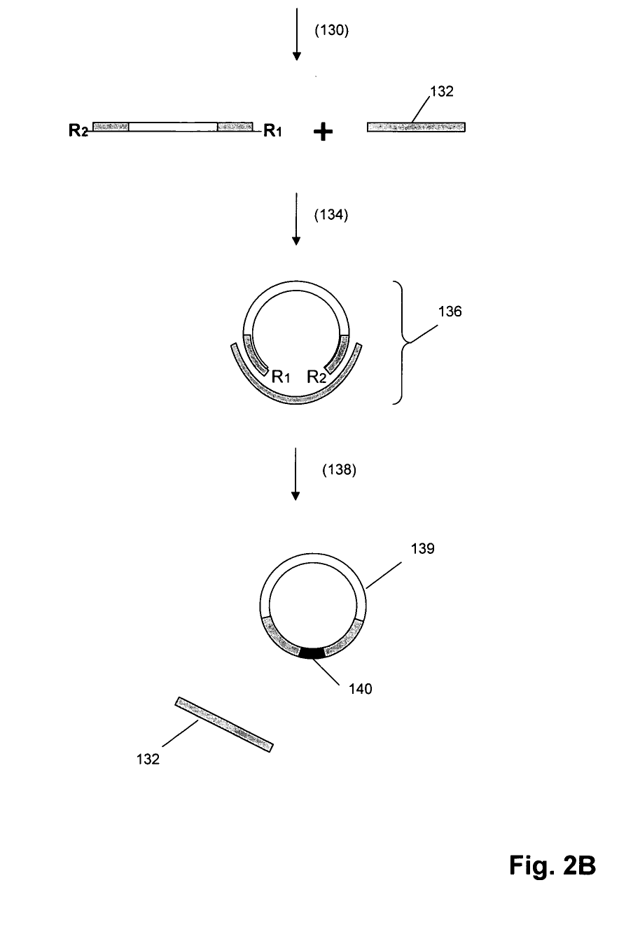

In another aspect, released signaling polynucleotides may be converted to a covalently closed circle to aid in its detection. As illustrated in FIG. 2A, in such as aspect, the initial steps of the method are the same as described above, with the exception that signaling reagent (200) has a signaling polynucleotide with the following additional features: At an end distal to cleavable linkage (112), there is a complementary functionality R₂ (202), first template-specific region (204), and second template-specific region (206). In one embodiment, complementary functionality R₂ (202) is a group attached to the 3′ carbon of the terminal nucleotide of signaling polynucleotide (212). As described more fully below, exemplary complementary functionalities include thiol groups and amino groups. As above, cleaving probe (103) and signaling reagents (200) are combined (116) with sample containing molecular complex (100) under conditions that permit the binding compounds to specifically bind to their respective epitopes, after which cleaving agents are induced to generate reactive species and cleave cleavable linkages within their effective proximities to release signaling polynucleotides (212). In this aspect, products resulting from the cleavage of cleavable linkage (112) include a released signaling polynucleotide (212) having a chemical group “R₁” (210) at the end of the cleavable linkage. Preferably, product “R₀” associated with the binding compound does not interfere with any subsequent processes in the assay. After signaling polynucleotides (212) are released, as shown in FIG. 2B, template oligonucleotide (132) is added (130) to the reaction mixture so that complex (136) forms in which first template-specific region (204) and second template-specific region (206) of released polynucleotide (212) are hybridized to their corresponding complementary regions oftemplate oligonucleotide (132). Preferably, template oligonucleotide (132) is present in substantial molar excess to released signaling polynucleotide (212). In various embodiments, such molar excess may be 5-fold, or 10-fold, or 100-fold over the expected concentration of released signaling polynucleotide. The lengths of first and second template-specific regions (204) and (206) are selected so that a stable duplex forms and as a consequence reactive functionality R₁ (210) and complementary functionality R₂ are forced into spatial proximity, so that the speed of their reaction together is increased. In one aspect, such length are in a range of from about 8 nucleotides to about 20 nucleotides, or from about 10 nucleotides to about 16 nucleotides. The two regions may have the same or different lengths. The total length of template oligonucleotide (132) is typically greater than or equal to the sum of the lengths of the first and second template-specific regions (204) and (206) depending on the nature of reactive functionality R₁ and complementary functionality R₂. If either R₁ or R₂ have linkages connecting them to their respective ends of signaling polynucleotide (212), then one or more nucleotides may be added between the segments of template oligonucleotide (132) complementary to first and second template-specific regions (204) and (206) in order to adjust the spatial alignment of R₁ and R₂ to maximize the chance of their reaction. After template-driven reaction (138), covalent linkage (140) is formed so that released signaling polynucleotide (212) becomes closed DNA circle (139). Template oligonucleotide (132) may be melted off prior to detection of closed DNA circle (139).

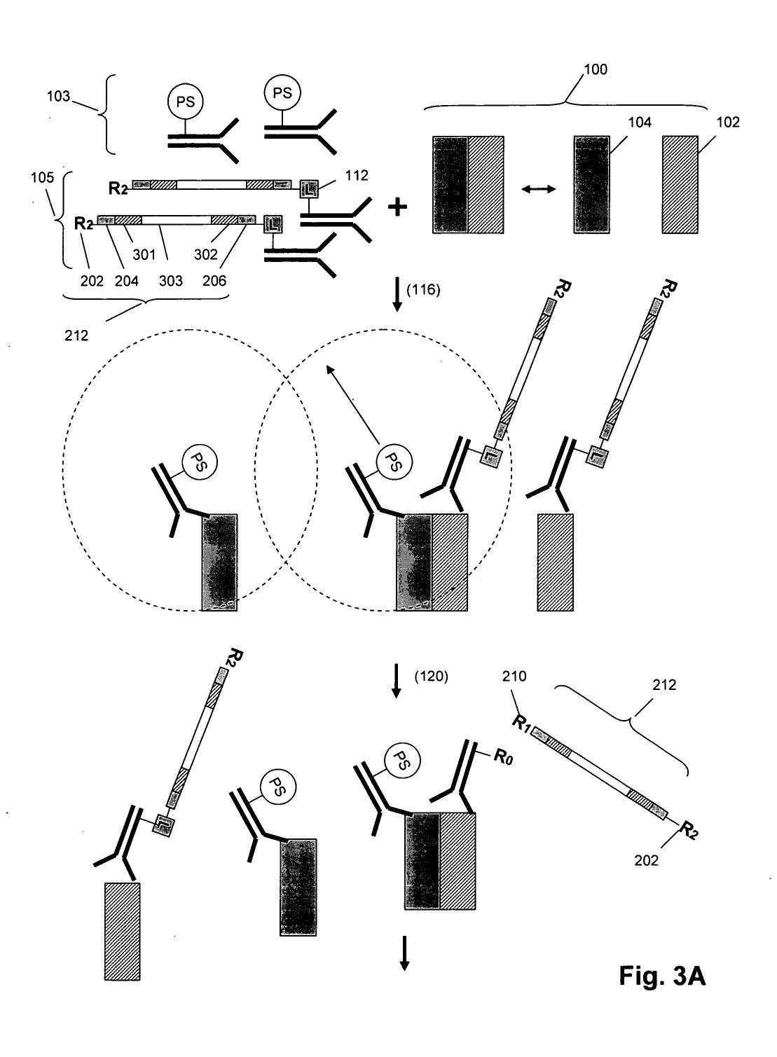

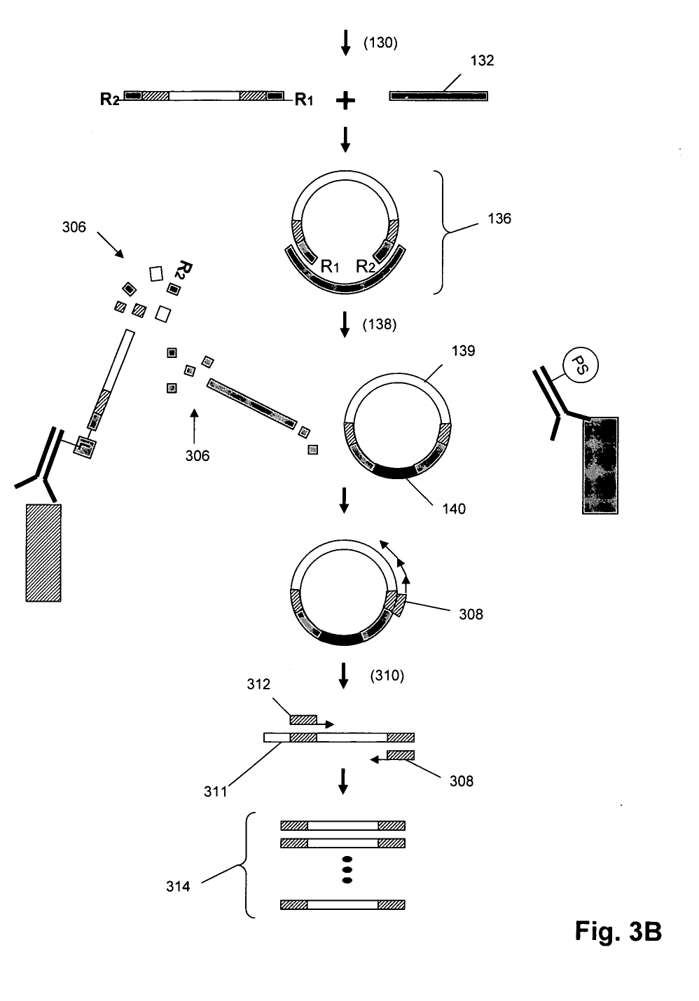

Another embodiment of the invention is illustrated in FIGS. 3A-3B. In this embodiment, the initial steps of the method are the same as described above, with the exception that signaling reagent (300) has a signaling polynucleotide with the following features: In addition to complementary functionality R₂ (202), first template-specific region (204), and second template-specific region (206), signaling polynucleotide (212) has first primer binding site (301), second primer binding site (302), and variable region (303). First and second primer binding sites (301) and (302) may be the same or different from, or may overlap, first and second template-specific regions (204) and (206), respectively. As illustrated in FIG. 3B, after formation of closed DNA circle (139) and melting of template oligonucleotide (132), the reaction mixture containing these components may be treated with an exonuclease that digests uncircularized signaling polynucleotides and free template oligonucleotide (1 32). An important feature of this embodiment is the selection of a complementary functionality that is not completely refractory to exonuclease digestion, such as thiol or amino groups. In any case, after exonuclease treatment, the exonuclease is inactivated, e.g. by heating, and primer (308) is added along with a polymerase and the appropriate deoxynucleoside triphosphates (dNTPs) for extending primer (308) to from an initial linear template (311). To such initial template (311), forward primers (312) is added for amplification, e.g. by PCR, to produce amplicon (314), which is then detected.

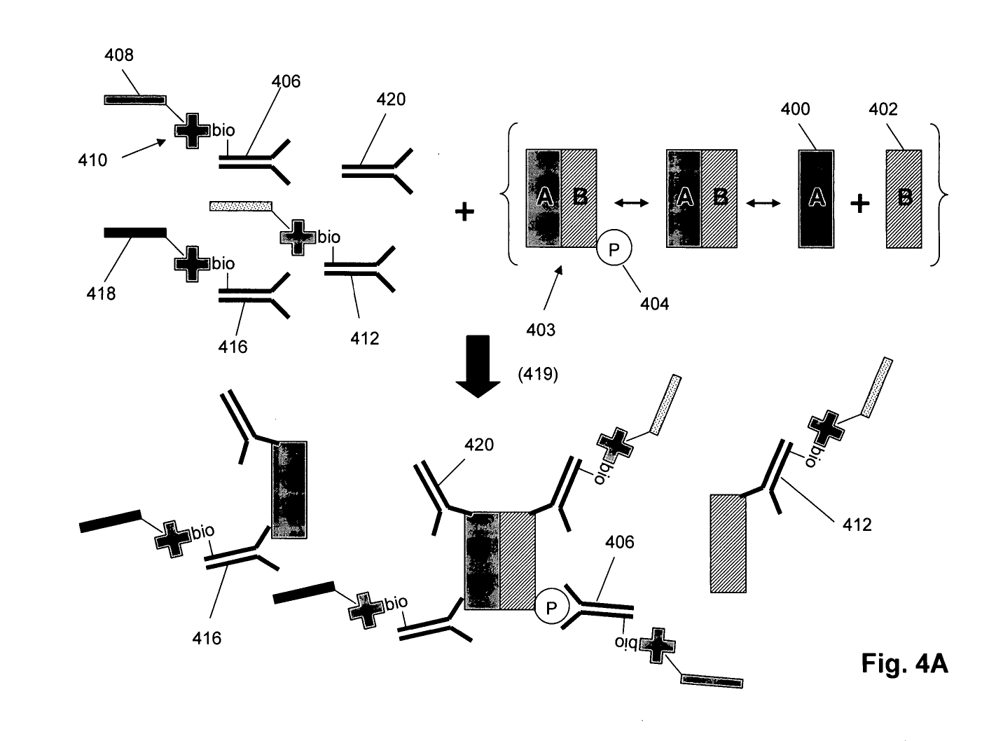

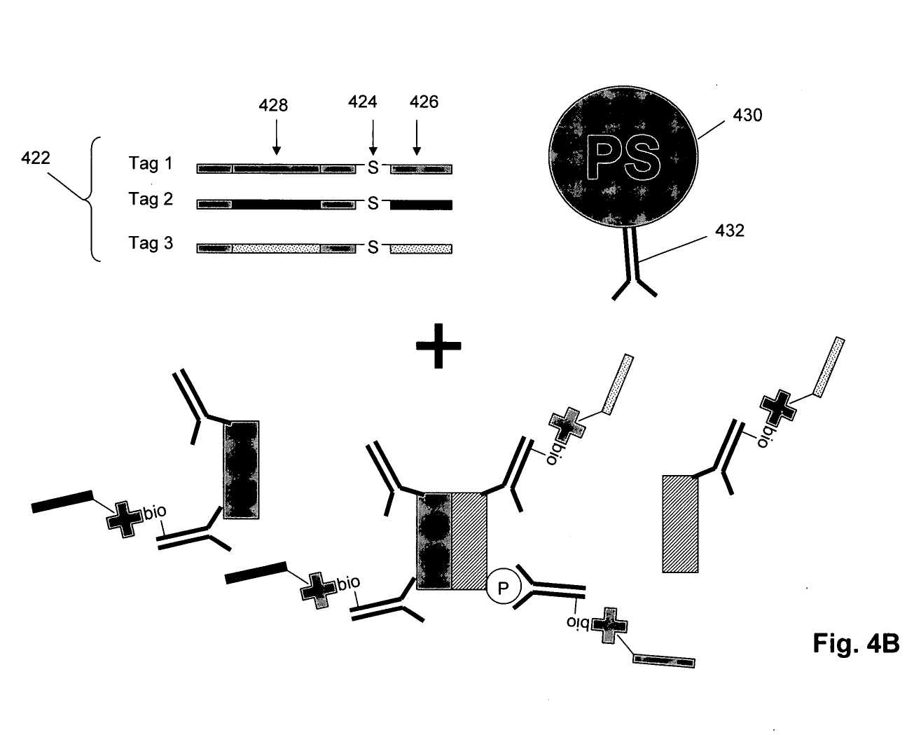

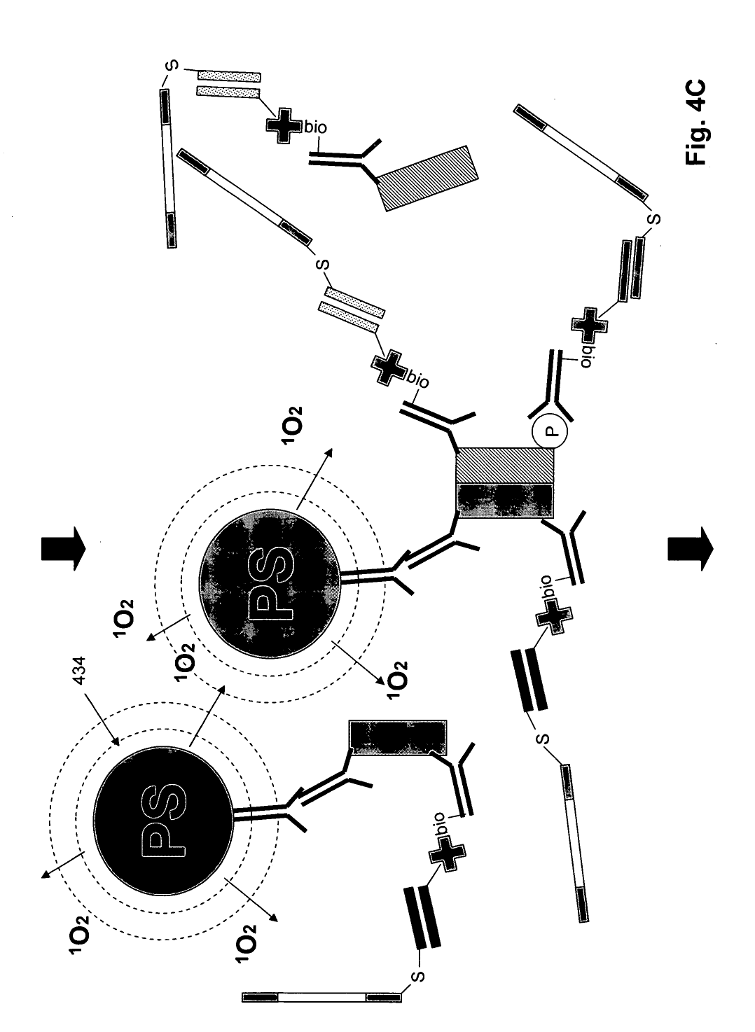

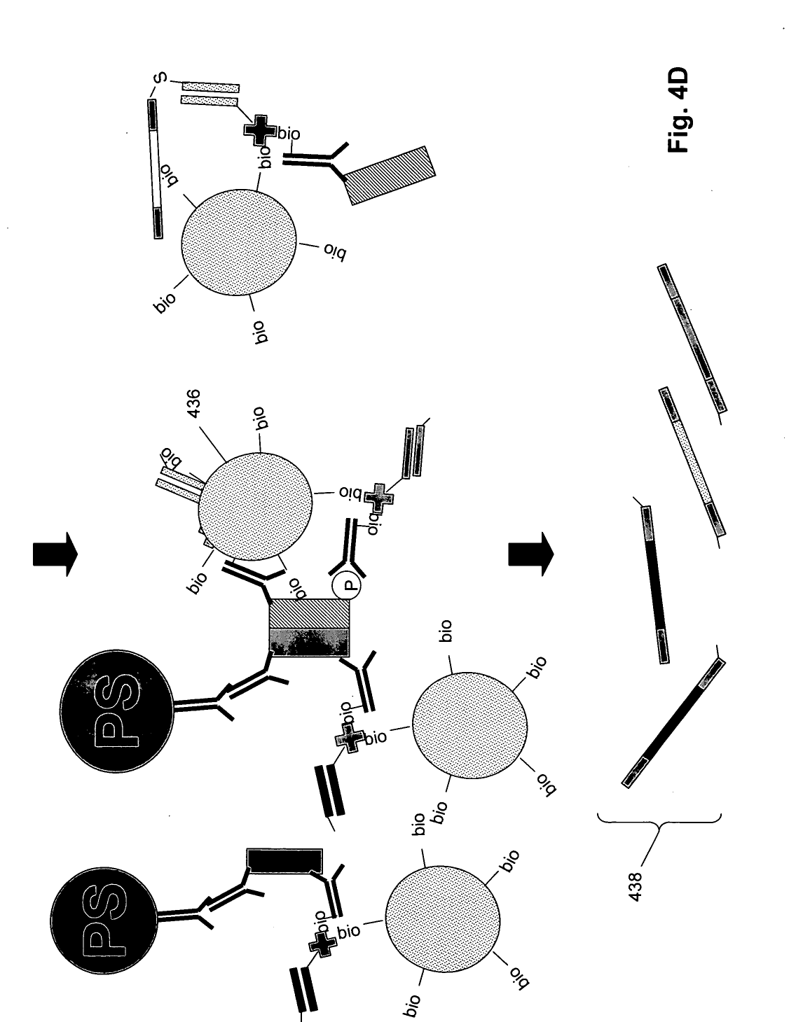

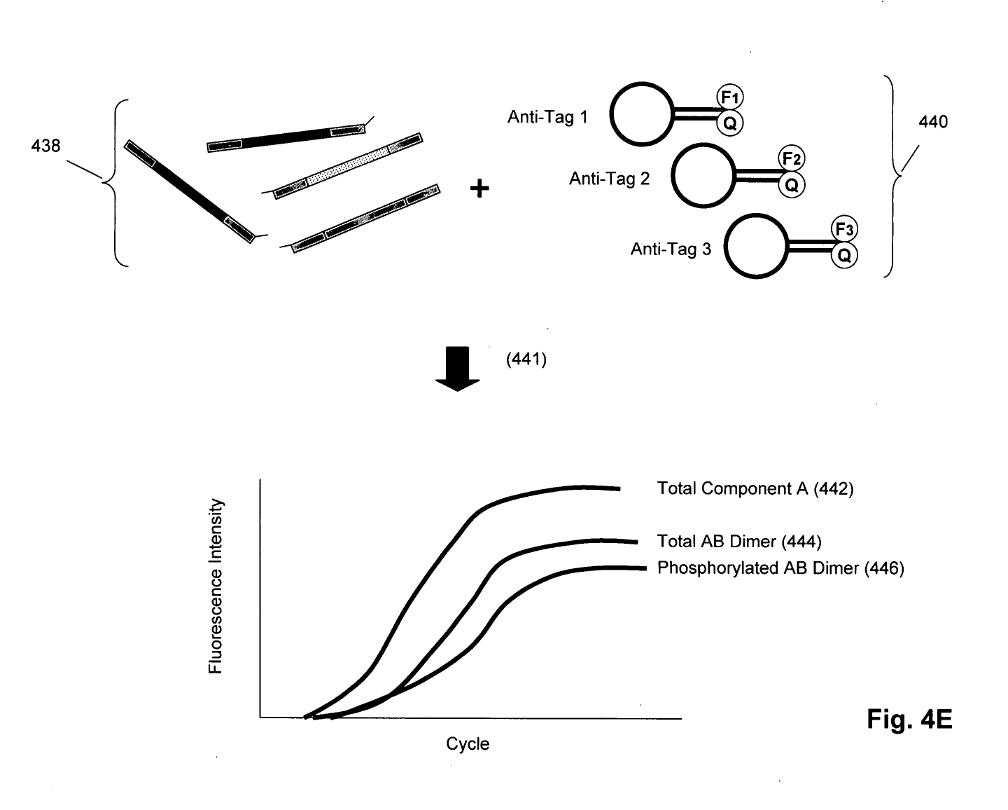

FIGS. 4A-4E illustrate another aspect of the invention in which oligonucleotide tags are attached to binding compounds via avidin/streptavidin-oligonucleotide conjugate, such as disclosed by Niemeyer et al, Nucleic Acids Research, 22: 5530-5539 (1994); and U.S. Pat. Nos. 5,561,043 and 5,965,133, which are incorporated by reference. Antibodies (406), (412), (416), and (420) are specific for separate epitopes on components A (400) and B (402) of phosphorylated complex AB (403), where a phosphate group is indicated by a “P” within a circle (404). Each of antibodies (406), (412), and (416) is conjugated via a biotin to a streptavidin derivatized with different oligonucleotide (408), (414), and (418), respectively. As discussed further below, oligonucleotides (408), (414), and (418) permit the attachment of a different oligonucleotide tag to each different antibody. Antibody (420) is a component of a cleaving probe, which in this embodiment comprises antibody (420) and a photosensitizer-impregnated bead (430) that has attached Fc-specific antibodies that (by appropriate selection of species-specific antibodies) bind only to antibody (420). These antibody reagents are combined (419) with a sample containing phosphorylated complex (403) to form in a reaction mixture containing several different complexes as shown. To this mixture are added (i) photosensitizer beads (430) having attached antibodies specific for the Fc region of antibody (420), and (ii) oligonucleotide tags (422) which each comprise an amplifiable segment (428) and an antibody-specific segment (426) linked by a cleavable linkage, such as a thioether bond (424). After incubation to form stable complexes, the photosensitizer beads are illuminated so that they generate singlet oxygen (434), which, in turn, cleaves the thioether linkages within the effective proximity of the beads to release oligonucleotide tags. After such cleavage, biotinylated solid supports, such as biotinylated beads, are added to bind all of the components in the reaction mixture that contain streptavidin, after which released oligonucleotide tags (438), which do not contain streptavidin, are separated or extracted from the mixture. Released oligonucleotide tags (438) may then be amplified and detected, e.g. by a real-time PCR using molecular beacons (440) specific for the different oligonucleotide tags. The different molecular beacons are detected via different fluorescent labels, F₁, F₂, and F₃, as disclosed in Vet et al, Proc. Natl. Acad. Sci., 96: 6394-6399 (1999), or the like. The respective fluorescent signals generated in the PCR correspond to total amount of component A (442), total amount of complex AB (444) (i.e. phosphorylated-plus non-phosphorylated complex AB), and amount of phosphorylated complex AB (446).

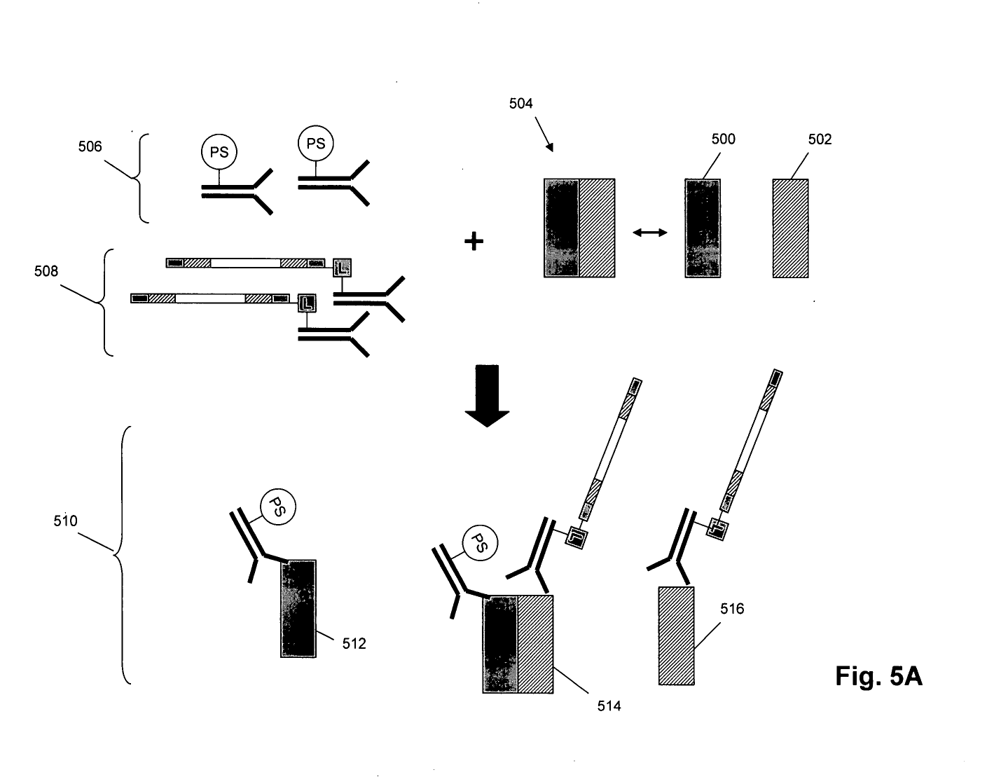

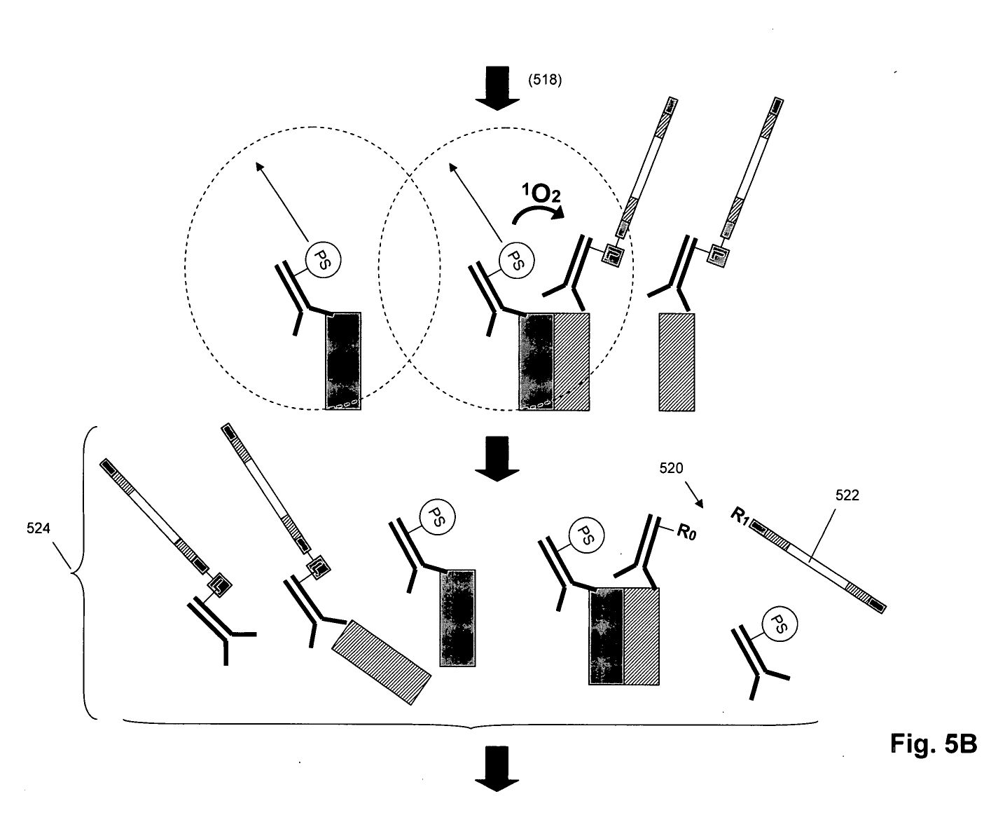

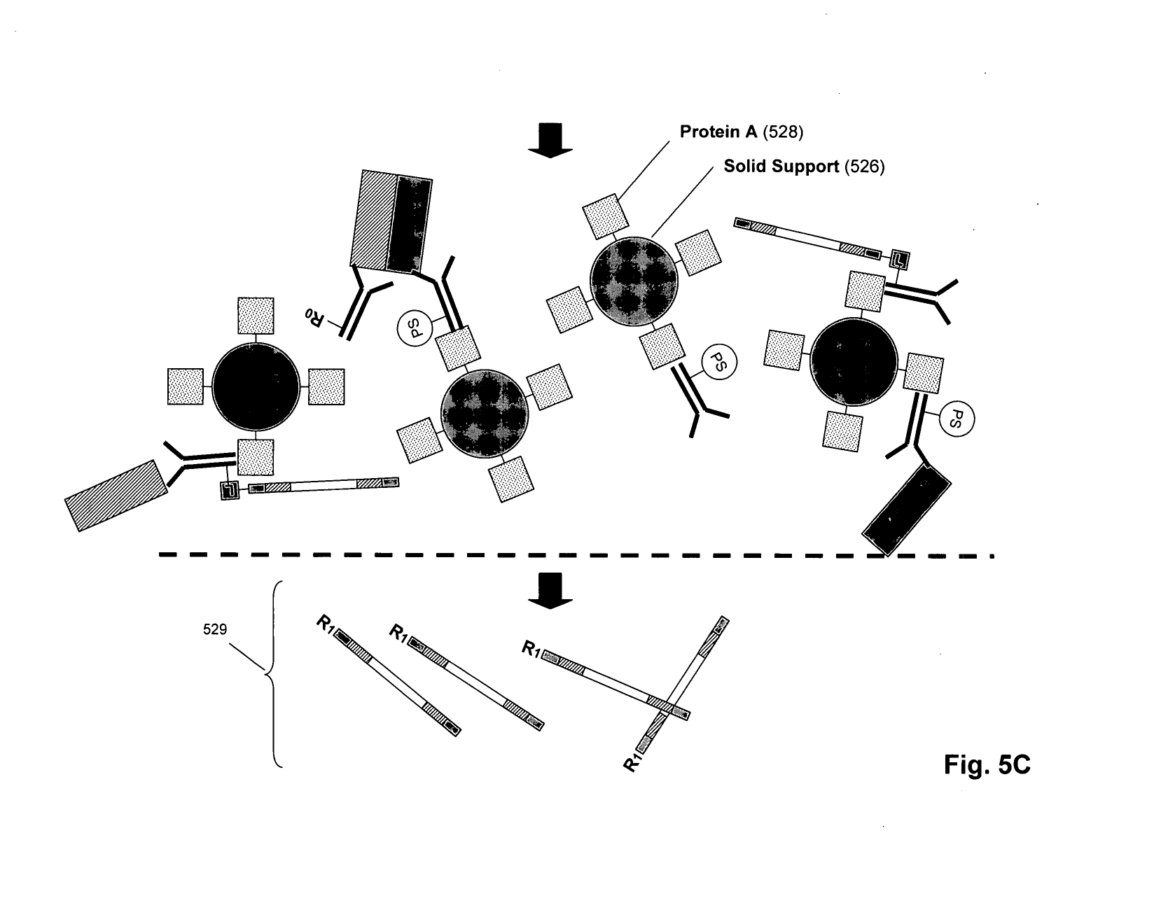

Another embodiment of the invention is illustrated in FIGS. 5A-5C. As above, cleaving probes (506) and antibody-oligonucleotide conjugates (508) are combined with a sample containing complex (504) that comprises components (500) and (502). Also, as described above, a reaction mixture is formed containing complexes (512), (514), and (516). After illumination (518) of photosensitizers to stimulate the generation of singlet oxygen, oligonucleotide tags (522) associated with complexes (514) are released into reaction mixture (524). After oligonucleotide tags are released, the reaction mixture is exposed to a solid support (526) with protein A or protein G, or like antibody binding compound, so that substantially all antibodies and complexes containing antibodies are bound. Use of protein A and/or G for affinity separation of antibodies is well-known, as evidence by the following references: Hahn et al, J. Chromatography B, 790: 35-51 (2003); Belenky et al, J. Immunoassay & Immunochemistry, 24: 311-318 (2003); Harlow and Lane, Antibodies: A Laboratory Manual (1988); Eliasson et al, J. Biol. Chem., 263: 4323-4327 (1988); Akerstrom et al, J. Biol. Chem., 261: 10240-10247 (1986); and the like. Solid phase support having protein A or protein G attached are available commercially, e.g. Amersham Biosciences (Piscataway, N.J.), Pierce Chemical Co. (Rockford, Ill.), Upstate Biotechnology (Charlottesville, Va.), and the like. The eluted release oligonucleotide tags (529) are amplified and measured as described in FIG. 4E.

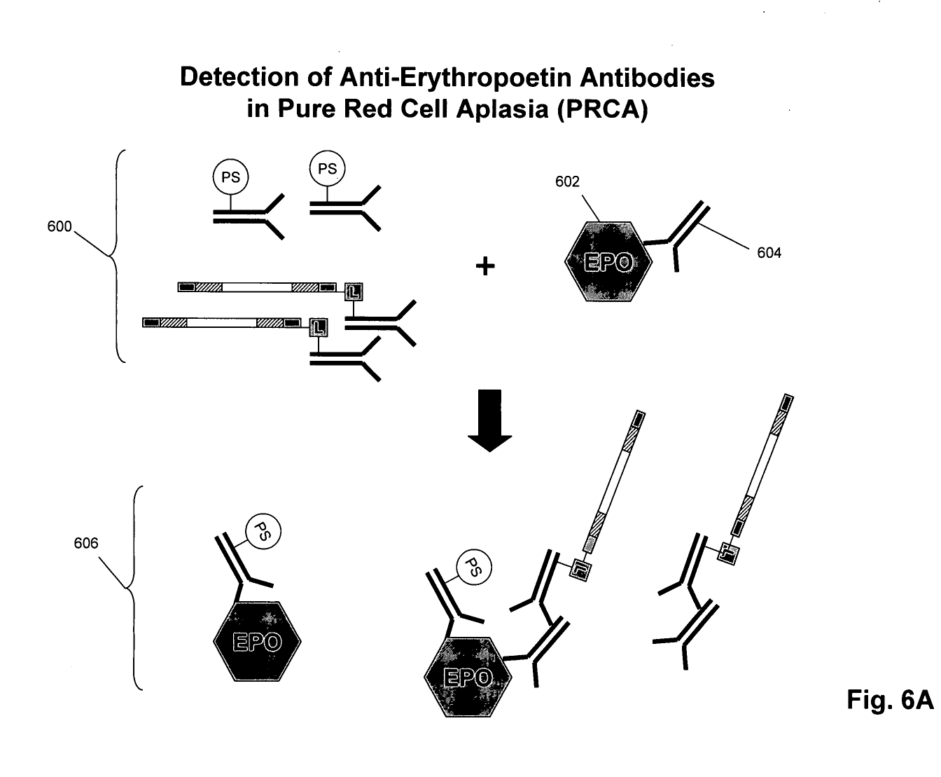

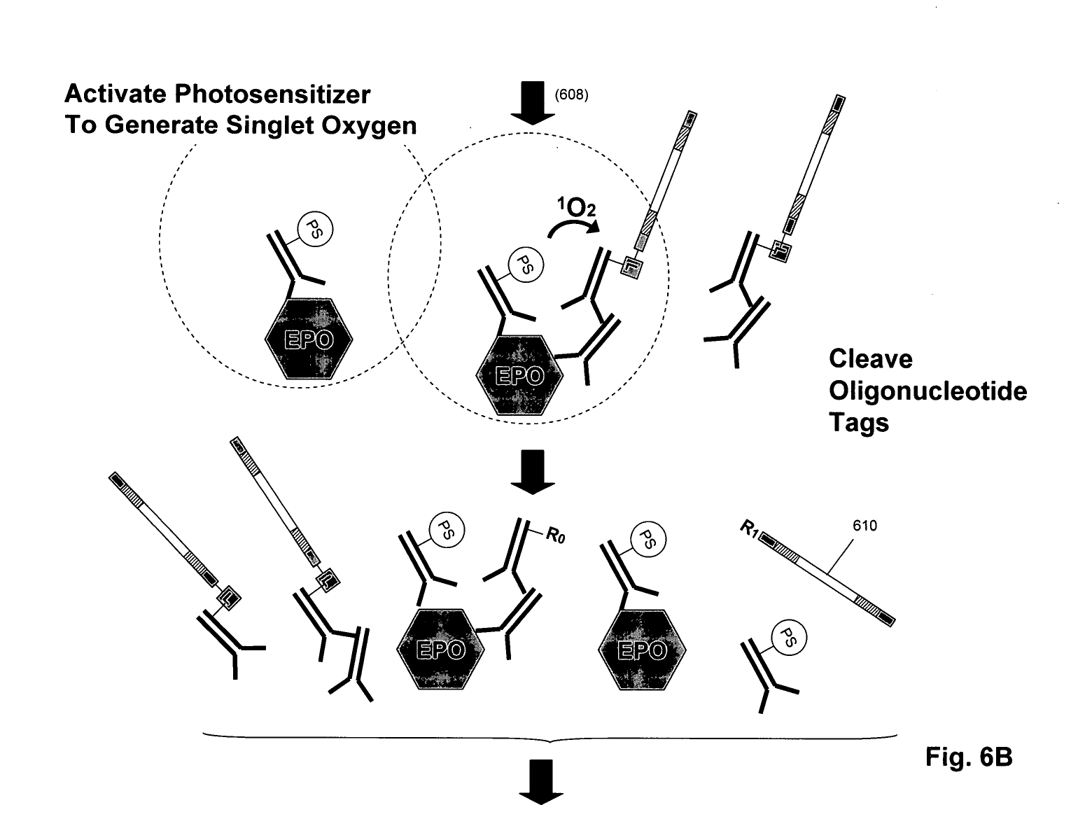

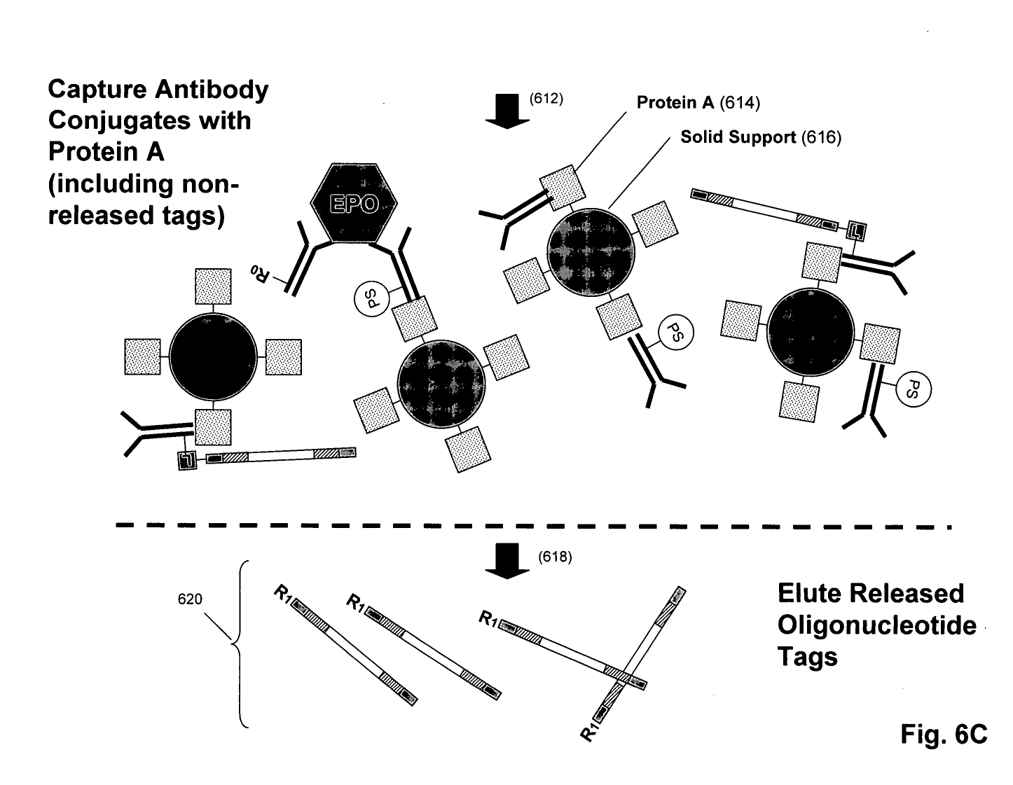

As mentioned above, in one aspect, the method of the invention can be used to detect antibodies specific for therapeutic proteins, such as erythropoietin, that are produced as an adverse reaction in some patients. As illustrated in FIG. 6A, antibody (604) binds to therapeutic protein, e.g. EPO, neutralizing its activity. To a patient's blood sample suspected of containing such complex is added reagents (600) comprising cleaving probes specific for an epitope of the therapeutic protein and binding compounds comprising an oligonucleotide tag linked via a cleavable linkage to an antibody specific for a common region of the patient's serum antibodies, such as an epitope in the IgG Fc region. In some instances, more than one such antibody can be employed to detect other antibody isotypes that may be involve in the neutralizing response, such as IgM antibodies, or IgE antibodies. After incubation under conditions that allow the formation of stable complexes (606), the reaction mixture is illuminated to stimulate the generation of singlet oxygen by photosensitizers of the cleaving probes, thereby releasing oligonucleotide tags (610). After such release, the reaction mixture is combined (612) with protein A or protein G, as appropriate, to bind all reaction components containing antibodies and to permit the released oligonucleotide tags to be removed (618), amplified, and identified as described in FIG. 4E, or by like methods.

In an assay, signaling polynucleotides are cleaved by a cleaving agent that reacts with the cleavable linkages to release the signaling polynucleotides from their respective binding compounds. The released signaling polynucleotides are then detected. A wide variety of cleavable linkages and corresponding cleaving agents may be employed in the invention. Whenever a homogeneous assay format is desired, preferably, the cleaving agent is a locally acting agent. That is, as explained more fully below, a cleavage-inducing moiety is employed in the assay that may be induced to create local conditions for cleavage of the cleavable linkages. In one aspect, such a cleavage-inducing moiety is a sensitizer that generates a reactive species, as described more fully below. Assays of the invention may also be conducted in a heterogeneous, or non-homogeneous, format. In such a format, binding compounds are combined with a sample in an assay reaction mixture so that the binding compound can specifically bind to their target molecules, or antigenic determinants, whenever they are available to form a stable complexes. Unbound binding compounds are then removed, or separated from, the stable complexes by washing, by filtration, centrifugation, magnetic separation, or the like. In a this format, cleavage of the signaling polynucleotides from the stable complexes need not be proximity dependent, since unbound binding compounds have been removed. Therefore, a larger variety of cleavage protocols can be used. Cleavage may still employ a sensitizer, as described above, to cleave an oxidatively labile linkage, but it may also employ various types of chemical, photochemical, or enzymatic cleavage of a variety of cleavable linking groups, such as are known in the art. For example, non-limiting examples of chemically cleavable linkages include disulfides (cleavable by reduction, typically using dithiothreitol), azo groups (cleavable with dithionate), sulfones (cleavable with basic phosphate, with or without dithiothreitol), glycols, cleavable by periodate, and esters, cleavable by hydrolysis. Photolabile linkers include, for example, azo linkages and o-nitrobenzyl ethers.

### Cleavable Linkages

Oligonucleotides are cleaved by a cleaving agent that reacts with the cleavable linkages to release the oligonucleotides from their respective binding compounds. A wide variety of cleavable linkages and corresponding cleaving agents may be employed in the invention. Whenever a homogeneous assay format is desired, preferably, the cleaving agent is a locally acting agent. That is, as explained more fully below, a cleavage-inducing moiety is employed in the assay that may be induced to create local conditions for cleavage of the cleavable linkages. In one aspect, such a cleavage-inducing moiety is a sensitizer that generates a short-lived active species, as described more fully below. Assays of the invention may also be conducted in a heterogeneous, or non-homogeneous, format. In such a format, binding compounds are combined with a sample in an assay reaction mixture so that the binding compound can specifically bind to their target molecules, or antigenic determinants, whenever they are available to form stable complexes. Unbound binding compounds are then removed, or separated from, the stable complexes by washing, by filtration, centrifugation, magnetic separation, or the like. In a this format, cleavage of the oligonucleotides from the stable complexes need not be proximity dependent, since unbound binding compounds have been removed. Therefore, a larger variety of cleavage protocols can be used. Cleavage may still employ a sensitizer, as described above, to cleave an oxidatively labile linkage, but it may also employ various types of chemical, photochemical, or enzymatic cleavage of a variety of cleavable linking groups, such as are known in the art. For example, non-limiting examples of chemically cleavable linkages include disulfides (cleavable by reduction, typically using dithiothreitol), azo groups (cleavable with dithionate), sulfones (cleavable with basic phosphate, with or without dithiothreitol), glycols, cleavable by periodate, and esters, cleavable by hydrolysis. Photolabile linkers include, for example, azo linkages and o-nitrobenzyl ethers. References describing many such linkages include Greene and Wuts, Protective Groups in Organic Synthesis, Second Edition (John Wiley & Sons, New York, 1991); Hermanson, Bioconjugate Techniques (Academic Press, New York, 1996); and Still et al, U.S. Pat. No. 5,565,324.

In one aspect, cleavable linkages of the invention are oxidation labile. In this aspect, cleavable linkages are preferably thioether or a selenium analog thereof; or olefin, which contains carbon-carbon double bonds, wherein cleavage of a double bond to an oxo group, releases the signaling polynucleotide. Illustrative thioether bonds are disclosed in Willner et al, U.S. Pat. No. 5,622,929 which is incorporated by reference. Illustrative olefins include vinyl sulfides, vinyl ethers, enamines, imines substituted at the carbon atoms with an α-methine (CH, a carbon atom having at least one hydrogen atom), where the vinyl group may be in a ring, the heteroatom may be in a ring, or substituted on the cyclic olefinic carbon atom, and there will be at least one and up to four heteroatoms bonded to the olefinic carbon atoms. The resulting dioxetane may decompose spontaneously, by heating above ambient temperature, usually below about 75° C., by reaction with acid or base, or by photo-activation in the absence or presence of a photosensitizer. Such reactions are described in the following exemplary references, which are incorporated by reference: Adam and Liu, J. Amer. Chem. Soc. 94, 1206-1209, 1972, Ando, et al., J.C.S. Chem. Comm. 1972, 477-8, Ando, et al., Tetrahedron 29, 1507-13, 1973, Ando, et al., J. Amer. Chem. Soc. 96, 6766-8, 1974, Ando and Migita, ibid. 97, 5028-9, 1975, Wasserman and Terao, Tetra. Lett. 21, 1735-38, 1975, Ando and Watanabe, ibid. 47, 4127-30, 1975, Zaklika, et al., Photochemistry and Photobiology 30,35-44, 1979, and Adam, et al., Tetra. Lett. 36, 7853-4, 1995. See also, U.S. Pat. No.5,756,726.

The formation of dioxetanes is obtained by the reaction of singlet oxygen with an activated olefin substituted with a signaling polynucleotide at one carbon atom and the binding moiety at the other carbon atom of the olefin. See, for example, U.S. Pat. No. 5,807,675. These cleavable linkages may be depicted by the following formula:
—W—(X)ₙC_{α}=C_{β}(Y)(Z)-
wherein:

W may be a bond, a heteroatom, e.g, O, S, N, P, M (intending a metal that forms a stable covalent bond), or a functionality, such as carbonyl, imino, etc., and may be bonded to X or C_{α}; at least one X will be aliphatic, aromatic, alicyclic or heterocyclic and bonded to C_{α} through a hetero atom, e.g., N, O, or S and the other X may be the same or different and may in addition be hydrogen, aliphatic, aromatic, alicyclic or heterocyclic, usually being aromatic or aromatic heterocyclic wherein one X may be taken together with Y to form a ring, usually a heterocyclic ring, with the carbon atoms to which they are attached, generally when other than hydrogen being from about 1 to 20, usually 1 to 12, more usually 1 to 8 carbon atoms and one X will have 0 to 6, usually 0 to 4 heteroatoms, while the other X will have at least one heteroatom and up to 6 heteroatoms, usually I to 4 heteroatoms;

Y will come within the definition of X, usually being bonded to C_{β} through a heteroatom and as indicated may be taken together with X to form a heterocyclic ring; Z will usually be aromatic, including heterocyclic aromatic, of from about 4 to 12, usually 4 to 10 carbon atoms and 0 to 4 heteroatoms, as described above, being bonded directly to C_{β} or through a heteroatom, as described above;

n is 1 or 2, depending upon whether the signaling polynucleotide is bonded to C_{α} or X; wherein one of Y and Z will have a functionality for attachment to a binding composition.

Illustrative cleavable linkages include S(signaling polynucleotide)-3-thiolacrylic acid, N(signaling polynucleotide), N-methyl 4-amino-4-butenoic acid, 3-hydroxyacrolein, N-(4-carboxyphenyl)-2-(signaling polynucleotide)-imidazole, oxazole, and thiazole.

Also of interest are N-alkyl acridinyl derivatives, substituted at the 9 position with a divalent group of the formula:
—(CO)X¹(A)-
wherein:

X′ is a heteroatom selected from the group consisting of O, S, N, and Se, usually one of the first three; and A is a chain of at least 2 carbon atoms and usually not more than 6 carbon atoms substituted with an signaling polynucleotide, where preferably the other valences of A are satisfied by hydrogen, although the chain may be substituted with other groups, such as alkyl, aryl, heterocyclic groups, etc., A generally being not more than 10 carbon atoms.

A list of exemplary cleavable linkages cleavable by singlet oxygen and their cleavage products are disclosed in U.S. patent publication US2003/0013126, which is incorporated herein by reference. The list includes thiazole cleavable linkage, “—CH₂-thiazole-(CH2)ₙ-C(═O)—NH—” (where n is in the range of from 1 to 12, and more preferably, from I to 6); oxazole cleavable linkage, “—CH₂-oxazole-(CH2)ₙ-C(═O)—NH—;” an olefin cleavable linkage; and thioether cleavable linkage having the form “—(CH₂₎₂—S—CH(C₆H₅)C(═O)NH—(CH₂)ₙ—NH—,” wherein n is in the range of from 2 to 12, and more preferably, in the range of from 2 to 6.

### Cleaving Agents

A cleaving agent is a moiety that produces an reactive species that is capable of cleaving a cleavable linkage, for example, by oxidation. Preferably, the reactive species is a chemical species that exhibits short-lived activity so that its cleavage-inducing effects are only in the proximity of the site of its generation. Either the reactive species is inherently short lived, so that it will not create significant background because beyond the proximity of its creation, or a scavenger is employed that efficiently scavenges the reactive species, so that it is not available to react with cleavable linkages beyond a short distance from the site of its generation. Illustrative reactive species include singlet oxygen, hydrogen peroxide, NADH, and hydroxyl radicals, phenoxy radical, superoxide, and the like. Illustrative quenchers for reactive species that cause oxidation include polyenes, carotenoids, vitamin E, vitamin C, amino acid-pyrrole N-conjugates of tyrosine, histidine, and glutathione, and the like, e.g. Beutner et al, Meth. Enzymol., 319: 226-241 (2000).

An important consideration for the cleaving agent and the cleavable linkage is that they not be so far removed from one another when bound to a target protein that the reactive species generated by the sensitizer diffuses and loses its activity before it can interact with the cleavable linkage. Accordingly, a cleavable linkage preferably are within 1000 nm, preferably 20-200 nm of a bound cleaving agent. This effective range of a cleaving agent is referred to herein as its “effective proximity.”

Generators of reactive species include enzymes, such as oxidases, such as glucose oxidase, xanthene oxidase, D-amino acid oxidase, NADH-FMN oxidoreductase, galactose oxidase, glyceryl phosphate oxidase, sarcosine oxidase, choline oxidase and alcohol oxidase, that produce hydrogen peroxide, horse radish peroxidase, that produces hydroxyl radical, various dehydrogenases that produce NADH or NADPH, urease that produces ammonia to create a high local pH.

A sensitizer is a compound that can be induced to generate a reactive intermediate, or species, usually singlet oxygen. Preferably, a sensitizer used in accordance with the invention is a photosensitizer. Other sensitizers included within the scope of the invention are compounds that on excitation by heat, light, ionizing radiation, or chemical activation will release a molecule of singlet oxygen. The best known members of this class of compounds include the endoperoxides such as 1,4-biscarboxyethyl-1,4-naphthalene endoperoxide, 9,10-diphenylanthracene-9,10-endoperoxide and 5,6,11,12-tetraphenyl naphthalene 5,12-endoperoxide. Heating or direct absorption of light by these compounds releases singlet oxygen. Further sensitizers are disclosed in the following references: Di Mascio et al, FEBS Lett., 355: 287 (1994) (peroxidases and oxygenases); Kanofsky, J. Biol. Chem. 258: 5991-5993 (1983) (lactoperoxidase); Pierlot et al, Meth. Enzymol., 319: 3-20 (2000) (thermal lysis of endoperoxides); and the like.

### Photosensitizers as Cleaving Agents

As mentioned above, the preferred cleaving agent in accordance with the present invention is a photosensitizer that produces singlet oxygen. As used herein, “photosensitizer” refers to a light-adsorbing molecule that when activated by light converts molecular oxygen into singlet oxygen. Photosensitizers may be attached directly or indirectly, via covalent or non-covalent linkages, to the binding agent of a class-specific reagent. Guidance for constructing of such compositions, particularly for antibodies as binding agents, available in the literature, e.g. in the fields of photodynamic therapy, immunodiagnostics, and the like. The following are exemplary references, which are incorporated by reference: Ullman, et al., Proc. Natl. Acad. Sci. USA 91, 5426-5430 (1994); Strong et al, Ann. New York Acad. Sci., 745: 297-320 (1994); Yarmush et al, Crit. Rev. Therapeutic Drug Carrier Syst., 10: 197-252 (1993); Pease et al, U.S. Pat. No. 5,709,994; Ullman et al, U.S. Pat. No. 5,340,716; Ullman et al, U.S. Pat. No. 6,251,581; McCapra, U.S. Pat. No. 5,516,636; and the like.

Likewise, there is guidance in the literature regarding the properties and selection of photosensitizers suitable for use in the present invention. The following exemplary references provide such guidance: Wasserman and R. W. Murray. Singlet Oxygen. (Academic Press, New York, 1979); Baumstark, Singlet Oxygen, Vol. 2 (CRC Press Inc., Boca Raton, Fla. 1983); and Turro, Modem Molecular Photochemistry (University Science Books, 1991).

The photosensitizers are sensitizers for generation of singlet oxygen by excitation with light. The photosensitizers include dyes and aromatic compounds, and are usually compounds comprised of covalently bonded atoms, usually with multiple conjugated double or triple bonds. The compounds typically absorb light in the wavelength range of about 200 to about 1,100 nm, usually, about 300 to about 1,000 nm, preferably, about 450 to about 950 nm, with an extinction coefficient at its absorbance maximum greater than about 500 M⁻¹ cm⁻¹, preferably, about 5,000 M⁻¹ cm⁻¹, more preferably, about 50,000 M⁻¹ cm⁻¹, at the excitation wavelength. The lifetime of an excited state produced following absorption of light in the absence of oxygen will usually be at least about 100 nanoseconds, preferably, at least about 1 millisecond. In general, the lifetime must be sufficiently long to permit cleavage of a linkage in a reagent in accordance with the present invention. Such a reagent is normally present at concentrations as discussed below. The photosensitizer excited state usually has a different spin quantum number (S) than its ground state and is usually a triplet (S=1) when the ground state, as is usually the case, is a singlet (S=0). Preferably, the photosensitizer has a high intersystem crossing yield. That is, photoexcitation of a photosensitizer usually produces a triplet state with an efficiency of at least about 10%, desirably at least about 40%, preferably greater than about 80%.

Photosensitizers chosen are relatively photostable and, preferably, do not react efficiently with singlet oxygen. Several structural features are present in most useful photosensitizers. Most photosensitizers have at least one and frequently three or more conjugated double or triple bonds held in a rigid, frequently aromatic structure. They will frequently contain at least one group that accelerates intersystem crossing such as a carbonyl or imine group or a heavy atom selected from rows 3-6 of the periodic table, especially iodine or bromine, or they may have extended aromatic structures.

A large variety of light sources are available to photo-activate photosensitizers to generate singlet oxygen. Both polychromatic and monochromatic sources may be used as long as the source is sufficiently intense to produce enough singlet oxygen in a practical time duration. The length of the irradiation is dependent on the nature of the photosensitizer, the nature of the cleavable linkage, the power of the source of irradiation, and its distance from the sample, and so forth. In general, the period for irradiation may be less than about a microsecond to as long as about 10 minutes, usually in the range of about one millisecond to about 60 seconds. The intensity and length of irradiation should be sufficient to excite at least about 0.1% of the photosensitizer molecules, usually at least about 30% of the photosensitizer molecules and preferably, substantially all of the photosensitizer molecules. Exemplary light sources include, by way of illustration and not limitation, lasers such as, e.g., helium-neon lasers, argon lasers, YAG lasers, He/Cd lasers, and ruby lasers; photodiodes; mercury, sodium and xenon vapor lamps; incandescent lamps such as, e.g., tungsten and tungsten/halogen; flashlamps; and the like. An exemplary photodiode for stimulating photosensitizers to generate singlet oxygen is a high power GaAlAs IR emitter LED, such as model OD-880W laser diode manufactured by OPTO DIODE CORP. (Newbury Park, Calif.).

Examples of photosensitizers that may be utilized in the present invention are those that have the above properties and are enumerated in the following references, which are incorporated by reference: Singh and Ullman, U.S. Pat. No. 5,536,834; Li et al, U.S. Pat. No. 5,763,602; Martin et al, Methods Enzymol., 186: 635-645 (1990); Yarmush et al, Crit. Rev. Therapeutic Drug Carrier Syst., 10: 197-252 (1993); Pease et al, U.S. Pat. No. 5,709,994; Ullman et al, U.S. Pat. No. 5,340,716; Ullman et al, U.S. Pat. No. 6,251,581; McCapra, U.S. Pat. No. 5,516,636; Thetford, European patent publ. 0484027; Sessler et al, SPIE, 1426: 318-329 (1991); Magda et al, U.S. Pat. No. 5,565,552; Roelant, U.S. Pat. No. 6,001,673; and the like.

As with sensitizers, in certain embodiments, a photosensitizer may be associated with a solid phase support by being covalently or non-covalently attached to the surface of the support or incorporated into the body of the support. In general, the photosensitizer is associated with the support in an amount necessary to achieve the necessary amount of singlet oxygen. Generally, the amount of photosensitizer is determined empirically. In one preferred embodiment, a photosensitizer is incorporated into a latex particle to form photosensitizer beads, e.g. as disclosed by Pease et al., U.S. Pat. No. 5,709,994; Pollner, U.S. Pat. No. 6,346,384; Pease et al, PCT publication WO 01/84157; and Chan-Hui et al, U.S. patent publication US2004/0229293, which references are incorporated by reference. In another exemplary embodiment, the photosensitizer Rose Bengal is covalently attached to 0.5 micron latex beads by means of chloromethyl groups on the latex to provide an ester linking group, as described in J. Amer. Chem. Soc., 97: 3741 (1975).

### Schemes for Template-Driven Circularization of Released Polynucleotides

A variety of schemes are available for creating covalent linkages between the ends of signaling polynucleotides by template-driven reactions. Chemistries for implement several such schemes are disclosed in the following references, which are incorporated by reference: Gryaznov et al, Nucleic Acids Research, 21: 1403-1408 (1993); Gryaznov et al, Nucleic Acids Research, 22: 2366-2369 (1994); Metelev et al, Nucleic Acids Research, 29: 4062-4069 (2001); Dolinnaya et al, Nucleic Acids Research, 21: 5403-5407 (1993); Gryaznov et al, J. Am. Chem. Soc., 115: 3808 (1993); Xu and Kool, Nucleic Acids Research, 27: 875-881 (1999); U.S. Pat. Nos. 5,681,943; 5,476,930; 5,426,180; and the like. In one aspect, an important feature of the invention is that a reactive functionality produced at the end of a signaling polynucleotide from the cleavage of a cleavable linkage be capable of participating as a reactant in a subsequent template-driven ligation reaction. Preferably, such reactive functionality is a thiol group. In another aspect, an important feature of the invention is that a complementary functionality be amenable to exonuclease digestion in a non-circularized state and be capable of participating in a template-driven ligation reation in a free state. The former property permits non-circularized signaling polynucleotide to be digested, thereby reducing substantially background signal, e.g. see Hardenbol et al (cited above). Such constraints suggest that preferred complementary functionalities be physically and chemically similar to the 3′ hydroxyl group of natural nucleosides. Preferably, complementary functionalities are selected from the group consisting of 3′ thiol groups and 3′ amino groups. In particular, complementary functionalities are HS— or H₂N— attached to the 3′ carbon of a signaling polynucleotide. Production of polynucleotides with such functionalities is disclosed in the following references, which are incorporated by reference: Gryaznov et al, Tetrahedron Lett., 34: 1261-1264 (1993); Gryaznov et al, Nucleic Acids Research, 20: 3403-3409 (1992); U.S. Pat. No. 5,932,718; Metelev et al, Nucleic Acids Research, 29: 4062-4069 (2001); Aubert et al, Nucleic Acids Research, 28: 818-825 (2000); Sabbagh et al, Nucleic Acids Research, 32: 495-501 (2004); Cosstick et al, Tetrahedron Lett., 30: 4693-4696 (1989); Xu and Kool, Nucleic Acids Research, 26: 3159-3164 (1998); Vyle et al, Biochemistry, 31: 3012-3018 (1992); and the like.

In one scheme, a signaling polynucleotide containing first and second primer binding sites and a 3′ thiol is synthesized on a conventional DNA synthesizer using standard phosphoramidite chemistry the phosphorothioamidite monomer disclosed in Vyle et al (cited above). In the final two coupling steps of the synthesis reaction, (i) an AminoLinker™ reagent (e.g. Applera Corporation or Glen Research) is coupled to the 5′ hydroxyl of the signaling polynucleotide to generate a free 5′ amino group after deprotection, and (ii) the free 5′ amino group is reacted with a conventional bifunctional reagent such as succinimidyloxycarbonyl-α-methyl-α-(2-pyridyidithio)toluene (SMPT); N-succinimidyl 3-(2-pyridyidithio)propionate (SPDP); or like reagents to generate a 5′ thiol group. These reagents and their application are disclosed in Hermanson, Bioconjugate Techniques (Academic Press, New York, 1996), which is incorporated by reference. After treatment with dithiothreitol (DTT), or like reagent, the free 5′ thiol may be linked to a free amine on a binding compound with another bifunctional linking reagent, such as SIAX, SIAC, GMBS, SMPB, SIAB, MBS, SMCC, or the like, which are disclosed in Hermanson (cited above). In each case, the resulting linkage contains a thioether moiety that may be cleaved with singlet oxygen to leave a free 5′ thiol on the released signaling polynucleotide. A free 3′ thiol group is produced by cleaving the signaling polynucleotide at the 3′ bridging phosphorothioate linkage using Ag+ or iodine in aqueous pyridine, as taught by Vyle et al (cited above). After such a signaling polynucleotide is cleaved, e.g. with exposure to singlet oxygen, a covalently closed circle is produced by forming a disulfide bond between the 5′ and 3′ thiol groups in a template-driven reaction. Preferably, a template oligonucleotide is added to an assay mixture is substantial excess to the released signaling polynucleotide, e.g. at least 10-fold excess, and more preferably, a 100-fold or 1000-fold excess. In the present scheme, such a template oligonucleotide may comprise the complements of the first and second primer binding sites separated by from 0-3 nucleotides. The template-driven reaction is carried out in the presence of an oxidant, such as KI₃ or K₃Fe(CN)₆, such as taught by Gryaznov and Letsinger, Nucleic Acids Research, 21: 1403-1408 (1993). Uncircularized signaling polynucleotides may be digested by exonuclease treatment, e.g. by a mixture of exonuclease I and exonuclease III, the latter of which will attach 3′ ends of uncircularized signaling polynucleotides that form duplexes with template oligonucleotides. Such treatment is disclosed in Hardenbol et al, Nature Biotechnology, 21″ 673-678 (2003); and Willis et al, U.S. Pat. No. 6,858,412, which references are incorporated by reference. Briefly, in a typical reaction 10 units exonuclease 1 and 200 units exonuclease III (U.S. Biochemical) in a 2 μL volume are added to a 10 μL assay mixture and incubated for 10-20 minutes at 37° C., 2 min at 95° C., and 1 min at 37° C.

Alternatively to the above, a 3′ thiol group may be provided by employing a signaling polynucleotide having a 3′-phosphorothioate group as taught by Gryaznov and Letsinger (cited above). Otherwise, the protocol of the above scheme is followed.

### Amplification and Detection of Circularized Polynucleotides

Circularized signaling polynucleotides may be amplified and detected by a variety of conventional techniques. In one aspect, a uracil may be inserted into the signaling polynucleotide to permit opening of the DNA circles by treatment with uracil-DNA glycosylase and a lyase or heat, followed by run-off synthesis of the linearized signaling polynucleotide in the presence of labeled dNTPs. In another aspect, circularized signaling polynucleotides may be amplified by PCR or NASBA, optionally after linearization by treatment with a reducing agent, such as DTT. Detection and/or quantification of amplicons generated by such amplification techniques are well known in the art, as disclosed in the many references cited above in the definitions of amplicon and polymerase chain reaction.

In one aspect, signaling polynucleotides may be detected by way of oligonucleotide tags, or barcode sequences, that provide a convenient means for multiplexing. For low degrees of multiplexing, such as from 2-6, multiplexing may be carried out with fluorescent dyes or quantum dots that may be employed in conjunction with a variety of readout platforms, including microarrays, conventional fluorimeters with multi-channel detection capabilities, e.g. SmartCycler (Cephied, Sunnyvale, Calif.), capillary electrophoresis, or the like. Such labeling techniques may be implemented by use of Taqman probes, molecular beacons, or the like. In one aspect, assays of the invention are multiplexed to include the detection of from 2-10 different signaling polynucleotides, or the detection of from 2-6 different signaling polynucleotides, or the detection of from 2-4 different signaling polynucleotides.

In another aspect, higher levels of multiplexing may be achieved using oligonucleotide tags to generated probes that bind to solid phase supports, such as microarrays, e.g. GeneFlex platform (Affymetrix, Santa Clara, Calif.), or to labeled microbeads, e.g. as disclosed in the references cited above in the definition of “microarray.”

### Non-Homogeneous Assay Formats

Exemplary non-heterogeneous assay formats are illustrated in FIGS. 4A-4E and FIGS. 5A-5C. In the example of FIGS. 4A-4E, biotinylated detection antibodies are labeled with streptavidin-oligonucleotide conjugates, such as described in Niemeyer et al, Nucleic Acids Research, 22: 5530-5539 (1994); Kukolka et al, Methods Mol. Biol., 283: 181-196 (2004); or the like, with the exception that the linkage between the streptavidin and oligonucleotide is not a thioether or other linkage susceptible to cleavage by singlet oxygen. Appropriate linkages are disclosed in Hermanson, Bioconjugate Techniques (Academic Press, New York, 1996). Each of the streptavidin-oligonucleotide conjugates is separately combined with an antibody so that there is a one-to-one correspondence between the specificity of the antibody and the identity of the oligonucleotide in a conjugate, this oligonucleotide being referred to herein as a “connector oligonucleotide.” Releasable oligonucleotide tags are then attached non-covalently by hybridization of corresponding complements of the connector oligonucleotides. Nucleotide sequences of the connector oligonucleotides are of course selected to be non-cross reactive with complements of the other connector oligonucleotides and to form duplexes of equivalent stability under assay conditions.

In the example of FIGS. 5A-5C, each detection antibody is conjugated via a thioether linkage to an oligonucleotide tag, e.g. as described in Ullman et al, Proc. Natl. Acad. Sci., 91: 5426-5430 (1994); Gullberg et al, Proc. Natl. Acad. Sci., 101: 8420-8424 (2004); and the like. Such conjugates are combined with cleaving probes and sample to form molecular complexes as shown in FIG. 5A, after which photosensitizers of the cleaving probes are activated to generated singlet oxygen. The single oxygen cleaves thioether bonds within the respective effective proximities of the cleaving probes to release oligonucleotide tags. To the reaction mixture are added solid supports derivatized with an immunoglobulin-binding compound, such as protein A or protein G. Such capture agents capture all complexes and/or conjugates containing immunoglobulin molecules. Released oligonucleotide tags are not captured and may be eluted away from the captured material for amplification and identification.

### Sample Preparation

Samples containing molecular complexes may come from a wide variety of sources including cell cultures, animal or plant tissues, microorganisms, patient biopsies, or the like. Samples are prepared for assays of the invention using conventional techniques, which may depend on the source from which a sample is taken. Guidance for sample preparation techniques can be found in standard treatises, such as Sambrook et al, Molecular Cloning, Second Edition (Cold Spring Harbor Laboratory Press, New York, 1989); Innis et al, editors, PCR Protocols (Academic Press, New York, 1990); Berger and Kimmel, “Guide to Molecular Cloning Techniques,” Vol. 152, Methods in Enzymology (Academic Press, New York, 1987); Ohlendieck, K. (1996). Protein Purification Protocols; Methods in Molecular Biology, Humana Press Inc., Totowa, N.J. Vol 59: 293-304; Method Booklet 5, “Signal Transduction” (Biosource International, Camarillo, Calif., 2002); or the like.

For blood specimens, the following references provide guidance for separating red blood cells from other cells in a specimen and for combining such other cells with immunomagnetic particles: Nakamura et al, Biotechnol. Prog., 17: 1145-1155 (2001); Moreno et al, Urology, 58: 386-392 (2001); Racila et al, Proc. Natl. Acad. Sci., 95: 4589-4594 (1998); Zigeuner et al, J. Urology, 169: 701-705 (2003); Ghossein et al, Seminars in Surgical Oncology, 20: 304-311 (2001); Terstappen et al, U.S. Pat. No. 6,365,362.

For biopsies and medical specimens, guidance for sample preparation is provided in the following references: Bancroft J D & Stevens A, eds. Theory and Practice of Histological Techniques (Churchill Livingstone, Edinburgh, 1977); Pearse, Histochemistry. Theory and applied. 4^{th} ed. (Churchill Livingstone, Edinburgh, 1980).

Samples are prepared for assays of the invention using conventional techniques, which may depend on the source from which a sample is taken.

Examples of patient tissue samples that may be used include, but are not limited to, breast, prostate, ovary, colon, lung, endothelium, stomach, salivary gland or pancreas. A tissue sample can be obtained by a variety of procedures including, but not limited to surgical excision, aspiration or biopsy. The tissue may be fresh or frozen. In one embodiment, assays of the invention are carried out on tissue samples that have been fixed and embedded in paraffin or the like; therefore, in such embodiments a step of deparaffination is carried out.

For mammalian tissue culture cells, fresh or frozen tissue specimens, or like sources, samples of complexes may be prepared by conventional cell Ilysis techniques (e.g. 0.14 M NaCl, 1.5 mM MgCl₂, 10 mM Tris-Cl (pH 8.6), 0.5% Nonidet P-40, and protease and/or phosphatase inhibitors as required).

### Exemplary Molecular Complexes

Below are various molecular complexes for detection by the method of the invention.

TABLE I
Exemplary Receptor Complexes of Cell Surface Membranes

| Dimer | Dimer |
|---|---|
| Her1-Her1 | IGF-1R-Her1 heterodimer |
| Her1-Her2 | IGF-1R-Her3 heterodimer |
| Her1-Her3 | Her1-PDGFR heterodimers |
| Her1-Her4 | Her3-PDGFR heterodimers |
| Her2-Her2 | Her2-P13K |
| Her2-Her3 | Her1-SHC |
| Her2-Her4 | Her3-SHC |
| Her3-Her4 | Her2-SHC |
| Her4-Her4 | Her3-P13K |
| Her2-PDGFR heterodimers | Her1-P13K |
| IGF-1R-Her2 heterodimer | VEGFR1(Flt1)-VEGFR2(KDR) |
| PDGFRα-PDGFRβ | |
| IGF-1R-Her2 heterodimer | IGF-1R-Her3 heterodimer |
| IGF-1R-PDGFR heterodimers | Her1-PDGFR heterodimers |
| Her2-PDGFR heterodimers | Her3-PDGFR heterodimers |
| αvβ3 heterodimer | αvβ5 heterodimer |
| Her1-P13K | Her2-P13K |
| Her3-P13K | Her1-SHC |
| Her2-SHC | Her3-SHC |
| IGF-1R-P13K | IGF-1R-SHC |
| IGF-1R-insulin receptor | PDGFR-P13K |
| PDGFR-SHC | IGF-1R-Her1 heterodimer |

The mechanisms of action of many drugs that are in use or are under development require the inhibition of one or more functions of receptor dimers, such as the association of component receptors into a dimer structure, or a function, such as an enzymatic activity, e.g. kinase activity, or autophosphorylation, that depends on dimerization. Such drugs are referred to herein as “dimer-acting” drugs. The number, type, formation, and/or dissociation of receptor dimers in the cells of a patient being treated, or whose treatment is contemplated, have a bearing on the effectiveness or suitability of using a particular dimer-acting drug. The following receptor dimers are biomarkers related to the indicated drugs.

TABLE II
Drugs Associated with Dimers of Cell Surface Membranes

| Dimer | Drug(s) |
|---|---|
| Her1-Her1, | Cetuximab (Erbitux), Trastuzumab (Herceptin), h- |
| Her1-Her2, | R3 (TheraCIM), ABX-EGF, MDX-447, ZD-1839 |
| Her1-Her3, | (Iressa), OSl-774 (Tarceva), PKI 166, GW2016, CI- |
| Her1-Her4, | 1033, EKB-569, EMD 72000 |
| Her1-IGF-R1 | |
| Her2-Her1, | 4D4 Mab, Trastuzumab (Herceptin), 2C4, GW2016 |
| Her2-Her3, | |
| Her2-Her2, | |
| Her2-Her4 | |
| VEGFR dimers | PTK787/K222584, ZD6474, SU6668, SU11248, |
| | CHR200131, CP547632, AG13736, |
| | CEP7055/5214, KRN633 |
| PDGFR dimers | SU6668, SU11248, AG13736, CHR200131 |
| FGFR dimers | CP547632, CHR200131 |

The following references describe the dimer-acting drugs listed in Table II: Traxler, Expert Opin. Ther. Targets, 7: 215-234 (2002); Baselga, editor, Oncology Biotherapeutics, 2: 1-36 (2002); Nam et al, Current Drug Targets, 4: 159-179 (2003); Seymour, Current Drug Targets, 2: 117-133 (2001); and the like.

TABLE III
Exemplary Protein-Protein Complexes in Apoptotic Pathways
(where “//” indicates a complex comprising the indicated proteins)

| | | | | |
|---|---|---|---|---|
| 14-3-3//BAD | BID//BAX | BAX//BAX | Bcl-X_{L}//BAD | Bcl-2//BAD |
| 14-3-3//BID | BID//BAK | BAX//Bcl-2 | Bcl-X_{L}//BIK | Bcl-2//BIK |
| NF-kB//1-kB | BID//Bcl-2 | Bcl-X_{L}//NIP3 | Bcl-X_{L}//BID | Bcl-2//BID |
| FADD//caspase-9 | BID//Bcl-X_{L} | Bcl-X_{L}//Nix | Bcl-X_{L}//Hrk | Bcl-2//Hrk |
| TRADD//caspase-9 | BID//A1/Bfl-1 | Bcl-2//Nix | Bcl-X_{L}//BIM | Bcl-2//BIM |
| Apaf-1//caspase-9 | Bcl-X_{L}//Bcl-G | Bcl-2//NIP3 | Bcl-X_{L}//Noxa | Bcl-2//Noxa |
| Bcl-2//NIP3 | Bcl-2//Bcl-G | Bcl-X_{L}//Puma | Bcl-X_{L}//Bmf | Bcl-2//Bmf |
| Bcl-2//Puma | | | | |

TABLE IV
Exemplary RTK Dimers and Intracellular Complexes

| RTK Dimer | Downstream Complexes |
|---|---|
| Her1-Her1 | Her1//Shc, Grb2//Sos, Her1//Grb7, Her1//RasGAP |
| Her1-Her2 | Her1//Shc, Grb2//Shc, Her2//Shc, Grb2//Sos, 14-3-3//Bad, |
| | Her1//RasGAP |
| Her1-Her3 | Her3//P13K, Her3//Shc, Her3//Grb7, Her1//Shc, Grb2//Sos, 14-3-3//Bad, |
| | Her1//RasGAP |
| Her1-Her4 | Her3//P13K, Her1//Shc, Grb2//Sos, Her1//RasGAP |
| Her2-Her2 | Her2//Shc, Grb2//Sos, 14-3-3//Bad, Her1//RasGAP |
| Her2-Her3 | Her3//P13K, Her3//Shc, Her3//Grb7, Grb2//Shc, Her2//Shc, Grb2//Sos, |
| | 14-3-3//Bad, Her1//RasGAP |
| Her2-Her4 | Her3//P13K, Grb2//Shc, Her2//Shc, Grb2//Sos, 14-3-3//Bad; YAP//Her4, |
| | Her1//RasGAP |
| Her3-Her4 | Her3//P13K, Her3//Shc, Her3//Grb7, YAP//Her4, Her1//RasGAP |
| Her4-Her4 | Her3//P13K, YAP//Her4, Her1//RasGAP |
| IGF-1R (covalent | IGF-1R//P13K, IGF-1R//Shc; IGFR//IRS1 |
| homodimers) | |
| VEGFR1(Flt1)- | VEGFR//Shc; VEGFR//P1(3)K; VEGFR//Src; VEGFR//FRS2 |
| VEGFR2(KDR) | |
| VEGFR2(KDR)- | VEGFR//Shc; VEGFR//P1(3)K; VEGFR//Src; VEGFR//FRS2 |
| VEGFR2(KDR) | |
| PDGFRa-PDGFRa | PDGFRa//Crk, PDGFR//Grb2; PDGFR//Grb7; PDGFRI/Nck; |
| | PDGFR//Shc;, PDGFR//STAT5 |
| PDGFRa-PDGFRb | PDGFRa//Crk, PDGFRb//GAP, PDGFR//Grb2; PDGFR//Grb7; |
| | PDGFR//Nck; PDGFR//Shc, PDGFR//Shp2; PDGFR//RasGAP, |
| | PDGFR//STAT5 |
| PDGFRb-PDGFRb | PDGFRb//GAP, PDGFR//Grb2; PDGFR//Grb7; PDGFR//Nck; |
| | PDGFR//Shc, PDGFR//Shp2, PDGFR//RasGAP;, PDGFR//STAT5 |
| Kit/SCFR(homodimers) | Kit//Shp-1; Kit//p85P1(3)K; Kit//Grb2; Kit//CRKL |
| FGFR (particularly FGFR1 | FGFR//PLCg1; FGFR//Crk; FGFR//FRS2; FGFR//Shp2; FGFR//Shb |
| homodimers) | |
| NGFR(TrkA)-NGFR(TrkA) | Trk//p75NTR; Trk//P1(3)K |
| | Shc//Grb2; Grb2//SOS Shc//Her1; Shc//Her2; Shc//Her3; P13K//Her1; |
| | IGF-1R//P13K; IGF-1R//Shc; Erk//Rsk; 14-3-3//FKHRL1; Cyclin |
| | D1//Cdk4; 14-3-3//tuberin; 14-3-3//Cdc25C; 14-3-3σ//Cdc2; |
| | RXRα//CAR; RXRα//PPARα; RXRα//PXR; Hsp90//Akt1 |

### Further Methods of the Invention

The present invention further includes a method of determining the presence and phoshorylation state of each of one or more phosphoproteins in a sample, which method comprises the steps of: (a) providing for each phosphoprotein a first binding compound specific for a first antigenic determinant and a second binding compound specific for a second antigenic determinant, the first and second binding compounds each having a distinct polynucleotide attached thereto by a cleavable linkage, and the second antigenic determinant including phosphate group such that a stable complex forms between the second binding compound and its respective phosphoprotein whenever the phosphate is present; (b) providing for each phosphoprotein a cleaving probe specific for a third antigenic determinant, the cleaving probe having a cleaving agent capable of generating a reactive species within an effective proximity, and the reactive species being capable of cleaving the cleavable linkage; (c) combining with the sample in an assay mixture the first and second binding compounds and the cleaving probes so that the first and second binding compounds and cleaving probes specifically bind to their respective antigenic determinants whenever present, and so that cleavable linkages are within the effective proximity of cleaving agents and are cleaved by the reactive species, and polynucleotides are released; and (d) determining the presence or phosphorylation state of the phosphoproteins by the released polynucleotide. In regard to the above method, the step of determining may include capturing said first and second binding compounds by a capture agent attached to a solid support so that said released polynucleotides can be eluted from the captured first and second binding compounds.

What is claimed is:

1. A method of determining the presence or amount of a molecular complex in a sample, the molecular complex comprising at least a first component and a second component, the method comprising the steps of:
providing a binding compound specific for the first component, the binding compound having a polynucleotide attached thereto by a cleavable linkage;
providing a cleaving probe specific for the second component, the cleaving probe having a cleaving agent capable of generating a reactive species within an effective proximity, and the reactive species being capable of cleaving the cleavable linkage;
combining with the sample in an assay mixture the binding compound and the cleaving probe so that the binding compound and cleaving probe specifically bind to the first and second components, respectively, and so that whenever the first component and the second component are in a molecular complex, the cleavable linkage is within the effective proximity of the cleaving agent and are cleaved by the reactive species, and the polynucleotide is released; and
determining the presence or amount of the molecular complex by the released polynucleotide.

2. The method of claim 1 further comprising the steps of: circularizing said released polynucleotide; and digesting uncircularized polynucleotide by exonuclease treatment.

3. The method of claim 2 wherein said polynucleotide comprises a first end, an oligonucleotide tag, a second end, and a complementary functionality at the first end or the second end, and said cleavable linkage being attached to the polynucleotide by an end opposite to that of the complementary functionality, and upon cleavage generating on such end a reactive functionality.

4. The method of claim 3 wherein said step of circularizing includes reacting said functionality with said complementary functionality in a template-driven reaction wherein said first end of the polynucleotide and said second end of the polynucleotide hybridize to a template oligonucleotide complementary thereto.

5. The method of claim 4 wherein said cleavable linkage is a thioether and said reactive species is singlet oxygen.

6. The method of claim 5 wherein said reactive functionality is a thiol group.

7. The method of claim 6 wherein said complementary functionality is a thiol group and wherein said template-driven reaction converts said reactive functionality and said complementary functionality into a disulfide linkage, thereby circularizing said released polynucleotide.

8. The method of claim 1 wherein said step of determining includes capturing said binding compounds by a capture agent attached to a solid support so that said released polynucleotide can be eluted from the captured binding compounds.

9. A composition comprising a plurality of binding compounds each having a polynucleotide attached by a cleavable linkage and each having a specificity for an antigen, such that each different binding compound has a different polynucleotide attached, and such that a binding compound having specificity for a different antigen has a different polynucleotide attached.

10. The composition of claim 9 wherein said cleavable linkage is selected from the group consisting of olefins and thioethers.

11. The composition of claim 10 wherein each said different antigen is a different protein in a molecular complex.

12. A composition comprising:
(a) one or more binding compounds each having a polynucleotide attached by a cleavable linkage and each having a specificity for a molecular complex, such that each different binding compound has a different polynucleotide attached, and such that a binding compound having specificity for a different antigenic determinant of the molecular complex has a different polynucleotide attached; and
(b) a cleaving probe specific for the molecular complex, the cleaving probe having a cleaving agent for generating an reactive species for cleaving the cleavable linkage.

13. The composition of claim 12 wherein said one or more binding compounds is a plurality of binding compounds, wherein said reactive species is singlet oxygen, and wherein said cleavable linkage is cleavable by singlet oxygen.

14. The composition of claim 13 wherein said polynucleotide has a complementary functionality on an end distal from said binding compound, and wherein upon cleavage said cleavable linkage generates a reactive functionality on an end of said polynucleotide proximal to said binding compound, such that the reactive functionality and the complementary functionality are capable of reacting in a template-driven reaction to form a stable linkage, thereby circularizing said polynucleotide.
